(19) United States
(12) Patent Application Publication
(10) Pub. No.: US 2014/0099328 A1
(43) Pub. Date: Apr. 10, 2014

(54) ANTIBODY PRODUCT COMPRISING N SPECIFIC ANTIBODIES

(71) Applicant: Jan Wesjohann, Visbek (DE)

(72) Inventor: Jan Wesjohann, Visbek (DE)

(73) Assignee: MAT-MALTA ADVANCED TECHNOLOGIES LIMITED, St. Julians (MT)

(21) Appl. No.: 14/110,044

(22) PCT Filed: Mar. 28, 2012
(86) PCT No.: PCT/EP2012/055456

§ 371 Date: Dec. 13, 2013

(30) Foreign Application Priority Data
Apr. 5, 2011 (DE) 10 2011 006 781.7

(51) Int. Cl. A61K 39/40

(52) U.S. Cl. CPC A61K 39/40
USPC 424/167.1; 424/164.1; 424/169.1

(57) ABSTRACT

Antibody product comprising n-specific antibodies
characterized in that
a) the n-specific antibodies in each case have an antibody content of at least 6/n % by weight of the total antibody component of the antibody product, and
b) 2, 3 or more of the n-specific antibodies target lipopolysaccharide-expressing microorganisms, and
c) the total amount of n-specific antibodies is 7% by weight of the total anti-body content of the antibody product.

### TECHNICAL FIELD

This invention relates to certain antibody products that include n-specific antibodies.

A vast array of therapeutic agents is currently available for the treatment of human diseases. Equally, a large number of prophylactic agents are in use. Despite the range of treatment options, however, there is a constant need for new therapeutic and prophylactic treatments, the development of new therapies, and the improvement and further development of established therapies.

The state of the art has shown that antibodies against endotoxins can be used in the treatment of certain diseases such as chronic pain syndromes. Endotoxins are decay products of bacteria that can trigger numerous physiological reactions in humans.

The state of the art has further shown that antibodies against endotoxins are found in the natural antibody spectrum of bovine colostrum.

In the context of this invention, bovine colostrum is used to refer to the first milk of mammals produced by the female mammary glands for optimal feeding newborn offspring during the first days of life. It is also called first milk, colostrum or animal milk (from cows) and consists of proteins, enzymes, vitamins, minerals, growth factors, amino acids and antibodies.

Many years of clinical experience of bovine colostrum in chronic pain syndromes have revealed serious shortcomings in the use of the available preparations: only a small number of patients benefited from highly effective treatment at an economically and biologically acceptable. A high proportion of patients experienced side effects such as milk or lactose intolerance.

Enrichment of anti-endotoxin antibodies in bovine colostrum by vaccination of pregnant cows was not viable for economic reasons.

In the state of the art there is a hyper-immunoglobulin preparation against the pathogen Pseudomonas aeroginosa for oral treatment and prophylaxis of typical bronchopulmonary infections in children with cystic fibrosis (CF). It is prepared on the basis of IgY, where the hens are vaccinated against Pseudomonas (E. Nillson et al, Pediatr Pulmonol., 2008 Aug. 4; E. Nillson et al, J Chromatogr B AnalytTechnol Biomed Life Sci, 2007, Sep. 1, 856 (1-2):75-80, Epub 2007 Jun. 2;. Kollberg H. et al, Pediatr Pulmonol, 2003 June, 35 (6):433-40).

The purpose of the present invention was therefore to make available therapeutic and prophylactic agents that are more effective than existing preparations and that preferably do not cause side effects through milk and lactose intolerance.

Another (partial) object of the present invention was to provide a method for preparing the therapeutic and prophylactic agents in accordance with the invention.

These objectives can be met by the subject matter of the independent claim or the claim process.

### SUMMARY OF THE INVENTION

What follows relates to the initial embodiment of the present invention, an antibody product comprising n-specific antibodies

where:
a) the n-specific antibodies in each case have an antibody content of at least 6/n % by weight of the total antibody component of the antibody product, and
b) 2, 3 or more of the n-specific antibodies target lipopolysaccharide-expressing microorganisms, and
c) the total amount of n-specific antibodies is ≧7% by weight of the total antibody content of the antibody product.

A further embodiment of the present invention relates to methods for producing an antibody product according to the present invention, comprising the following steps:
a) immunizing n groups of animals with only one micro-organism species each and/or a part of the only one microorganism species, where each n group is given a different microorganism species and/or part of the different micro-organism species, and where at least 2 of the micro-organism species and/or parts of the microorganism species are lipopolysaccharide-expressing micro-organism species or are derived therefrom.
b) obtaining an antibody-containing fraction from each of the n groups
c) mixing the antibody fractions
d) if necessary, concentrating the antibody content in the antibody fractions and/or in the mixture of antibody fractions.

### DETAILED DESCRIPTION OF THE INVENTION

Surprisingly, our own studies showed that an antibody product according to the present invention is suitable for the treatment and prophylaxis of many diseases and in many cases improved the treatment and prophylaxis of many diseases.

The studies further showed that many diseases, especially chronic ones, are influenced by a common mechanism.

Surprisingly, the studies showed that these diseases can be at least partially caused and prolonged by a faulty biological barrier against bacterial toxins, especially endotoxins. In addition to the faulty mechanical barrier function of the mucous membranes of the digestive tract (Goebel A. et al., Rheumatology, 2008), the faulty recognition of toxins as antigens by immune cells (Waaga-Gasser A M, et al, International Journal of Clinical Pharmacology and Therapeutics, In 2009) is a further precondition for the emergence of the symptoms of these diseases.

However, the background of the state of the art did not suggest that the antibody products according to the invention could improve treatment and prophylaxis of many diseases.

The common mechanism has shown that the incorrect processing of bacterial antigens is due to the fact that the antigen-host immune cells of the mucous membranes (especially in monocytes) do not have a sufficient degree of “organized cell death”, i.e. sufficient apoptosis is not triggered. Apoptosis normally leads to a local neutralization of the toxins within the immune barrier of the digestive tract.

When compared with patients with intact barrier function, patients with defective mechanical and immunological barrier function have an excess of venous blood immune cells, which continue to introduce bacterial toxins from the digestive tract. These immune cells do not undergo apoptosis in the normal way once toxins have been absorbed. In close correlation to this finding, the cellular immune system contains a set of defective humoral immune responses in patient serum or plasma that is typical of this disease mechanism.

Surprisingly, the studies showed that the antibody products according to the invention had far greater therapeutic and/or prophylactic effect than existing preparations in patients with one or more specific diseases. The patients in the studies suffered from an idiopathic pain syndrome as well as one or more other diseases (co-morbidities).

The results of the clinical studies carried out using antibody products corresponding to an embodiment of this invention are also the first to demonstrate therapeutic effects on comorbidities in study patients. Surprisingly, this is also the first time that is has been shown that endotoxins may play a pathogenic role not only in triggering and prolonging chronic (previously: idiopathic) pain syndromes but also in terms of the typical symptoms of known diseases.

One embodiment of the present invention is an antibody product comprising n-specific antibodies

characterized in that:
a) the n-specific antibodies in each case have an antibody content of at least 6/n % by weight of the total antibody component of the antibody product, and
b) 2, 3 or more of the n-specific antibodies target lipopolysaccharide-expressing microorganisms, and
c) the total amount of n-specific antibodies is ≧7% by weight, preferably ≧8% by weight, more preferably ≧10% by weight, most preferably ≧15% by weight of the total antibody content of the antibody product.

Surprisingly, an antibody product according to the invention has greater therapeutic and/or prophylactic effect than currently known preparations.

Antibody products according to the invention have further advantages: they cause fewer side effects than conventional therapies, while a high degree of efficacy is maintained when using antibody products according to the invention. This is particularly true of the preferred embodiments described below.

Another positive effect to treatment with antibody products according to the invention is an improvement in the mood and quality of sleep of the patients in question. It was also shown that antibodies could often support healing processes.

Moreover, antibody products according to the invention do not cause side effects from milk and lactose intolerance.

In the context of the present invention, an antibody or antibody portion is a protein from the class of globulins with at least one specific antigen-binding site (paratope). Antibodies are formed in vivo in response to specific antigens.

Antigens are substances that cause a specific immune response in human and animal organisms, resulting inter alia in the formation of antibodies.

An antigen can have several epitopes (antigenic determinant, antigen-binding site) that can bind to different antibodies. That is why, in vivo, it forms a mixture of antibodies of different specificities (polyclonal antibodies), even when immunized with a single antigen. Conversely, monoclonal antibodies are said to be those that have uniform mono- or bi-specificity.

A specific antigen usually induces the formation of only a few, very specific, matching antibodies that recognize only the foreign substance through specific, non-covalent bonding.

In this text the word “antigen” refers mainly to microorganisms (species) or parts thereof.

Lipopolysaccharides are compounds of fat-like (lipo) components and sugar components (polysaccharides). They can be found in the outer membrane of Gram-negative bacteria and act as antigens. As the bacteria decay, parts of the lipopolysaccharide separate off and become toxic. These parts are referred to as endotoxins.

Under cross-reactivity conditions, the antibody binds to two different antigens that have an identical or similar binding site (epitope). In the production of antibodies an antigen with a variety of epitopes might be used to give a mixture containing different antibodies. When using this antibody mixture, the antibodies react under cross-reactivity conditions, not only against the original antigen but also against antigens from other sources.

The determination of parameters in the context of the invention is set out below.

1.) Determining the total antibody content of an antibody product:

The total antibody content of an antibody product can be determined using widely available Kit Systems. The “ChickenIgG ELISA Quantitation Kit from Bethyl Laboratories Inc. is particularly suitable for determining the total IgG fraction of a formulation. The kit can be adjusted routinely to determine, for example, the total IgA or IgY content (etc.) of a formulation. In such cases it may be preferable to determine the respective proportions antibodies (IgA, IgG, IgMetc) separately and add them together to determine the total antibody content.

2.) Determining n:
I. Determination of a number (x) of antigens that will be targeted by an antibody in an antibody product, where each antibody component against an antigen is ≧0.5% by weight of the total antibody content of the antibody product.
   It is generally useful to carry out this step separately for each antigen, and preferably for each microorganism species.
II. An exemplary, preferred determination of the proportion of antibodies that target one of the (x) antigens according to I., based on the total antibody content of the antibody product:
   For determinations I and II, a sample of the antibody product will be passed over a column (or batch) prepared with a selected antigen that is in excess relative to the total antibody content of the antibody product. Conditions must be selected such that, in general, only specific antibody binding takes place. An antigen that binds ≧0.5% of antibodies as a proportion of the total amount of the antibody content of the antibody product is used to determine the number (x). An antigen that binds <0.5% by weight of the antibodies as a proportion of the total amount of the antibody content of the antibody product is not taken into account to determine the number (x).
   II. is used to determine the individual proportion of an antibody in the antibody product that is directed against an antigen of (x) antigens by % by weight, based on the total amount of antibody content of the antibody product.
III. Using an iterative process:
   Variant a): Each antibody component that is directed against an antigen of (x) antigens is equal to ≧6/(x) % by weight of the total antibody content of the antibody product. Then (x)=n.
   Variant b): One or more antibody components each directed against a different antigen of (x) antigens are equal to <6/(x)% by weight of the total antibody content of the antibody product.
      The number (x) of antigens to be used is then reduced by the number (y) of antibody component(s) that account for <6/(x)% by weight of the total antibody content of the antibody product.
      Now check the antibody components each targeting a different antigen of (x−y) antigens to ensure that each antibody component targeting each different antigen of (x−y) antigens is equal to ≧6/(x−y)% of the total antibody content of the antibody product:
         1. In this case, then (x−y)=n.
         2. If this is not the case, then repeat variant b) until each antibody component targeting an antigen of (x) antigens is equal to ≧6/(x) % by weight of the total amount of antibody content of the antibody product.

3.) Determining the total proportion of n-specific antibodies in the total antibody content of the antibody product:

To determine the total proportion of n-specific antibodies in the total antibody content of the antibody product, add the % by weight values of n-specific antibodies under 2.) II. The total % by weight values of these n-specific antibodies is the total proportion of n-specific antibodies as a % by weight of the total antibody content of the antibody product.

With regard to the determination of parameters in the context of this invention, it is further preferred:

4.) Preferred determination of total content of n-specific antibodies:

In the context of the present invention, the total content of n-specific antibodies as a proportion of the total amount of antibodies in the antibody product is determined once the value of n according to 2), preferably n according to 5) (see below), more preferably n according to 6.) (see below) have been established, and the antibody product sample has been passed over a column (or batch) prepared with all antigens targeting n-specific antibodies, where the antigens are in excess relative to the total antibody content of the antibody product. The conditions ensure that, generally speaking, only specific antibody binding takes place. This determines the proportion of bound antibodies in the antibody product (in % by weight) based on the total amount of antibody content of the antibody product.

The advantage of the preferred method of determining the total n-specific antibody component as a proportion of the total antibody fraction of the antibody product is that it allows for the possibility of cross-reactivity occurring (binding one of the n-specific antibodies of the antibody product by passing a sample of the antibody product over different columns, each prepared with a selected antigen (according to 2) II). The total of the % by weight values according to 2.) II. gives a higher total proportion of n-specific antibodies after 3.) as a proportion of the total antibody content of the antibody product, when compared with the preferred method of determination described here.

5.) Taking account of possible cross-reactivity when determining n:

The determination of each proportion of n-specific antibodies targeted against (x) antigens according to 2.), as a proportion of the total antibody content of the antibody product, should preferably take account of possible cross-reactivity.

I, II and III can then be established from the determination of n according to 2.).
a. For evidence of cross-reactivity, the first column (or batch) with all except one of the antigens targeted by n antibodies according to 2.) I, II and III should be prepared, with the antigens in excess relative to the total antibody content of the antibody product. A second column (or batch) is then prepared with the single remaining antigen.
b. A sample of the antibody product is passed over the first and second columns in succession. The conditions ensure that, generally speaking, only specific antibody binding takes place. Each bound antibody component as a % by weight of the total antibody content of the antibody product is determined on the second column.
c. The process is carried out with all combinations of antigens targeted against n antibodies.
d. If in each process an antibody component of ≧0.5% of the total antibody component of the antibody product is bound in the second column, n does not change.
e. If in one or more of the processes each of the antibody components that is bound to the antigen of the second pillar is <0.5% by weight of the total amount of antibody content of the antibody product, then cross-reactivity relevant to the invention is occurring. The antigen with the lowest antibody component and a proportion of <0.5% of the total antibody content of the antibody product bound to the second column is not considered for n: n is reduced by a value of 1 (=n−1).
f. This step should be repeated with a first column (or batch) prepared with all except one of the antigens against which the (n−1) antibodies are targeted, and each antigen should be in excess relative to the total antibody content of the antibody product. Thus the first column is not loaded with the antigen that, in the first process, bound <0.5% by weight of antibodies and the lowest proportion of antibodies as a total of the antibody content of the antibody product, nor prepared with any other antigen against which the (n−1) antibodies are prepared. A second column (or batch) is then prepared with the single remaining antigen set aside during this process.
   A sample of the antibody product is passed over the first and second columns in succession. The conditions ensure that, generally speaking, only specific antibody binding takes place. Each bound antibody component is determined as % by weight of the total antibody content of the antibody product.

Steps c, d, e and f can be repeated as often as required mutatis mutandis until n is no longer variable.

Once n has been established according to a to f, it is necessary to check whether n is valid for all of them, and whether each of the n-specific antibodies has a proportion of ≧6/n % by weight of the total antibody content of the antibody product. If this is not the case, use the method described in 2.) III. variant b) mutatis mutandis. To determine whether the criteria ≧6/n % of the total antibody content of the antibody product has been met, the values obtained for each antibody according to 2.) I. are used.

With regard to the determination of parameters in relation to the invention, it is particularly preferable to take account of possible cross-reactivity in the calculation according to 2.) III.

6.) Taking account of possible cross-reactivity according to 2.) III:

Context: An antigen that according to 2.) I and II binds ≧0.5% by weight of the antibodies as a proportion of the total amount of the antibody content of the antibody product, is taken into account when calculating (x) or n according to 2.) III.

It is preferable, however, to use the value for n determined according to 5.) when taking account of possible cross-reactivity. To determine whether the proportion of the total antibody content of the antibody product is ≧6/n % by weight, the values obtained for each antibody according to 5.) b and c in the most recent iteration stage will be used.

7.) Determining the number (a) of antibodies targeted against LPS-expressing organisms in parameter b) [“2, 3 or more of the n-specific antibodies are targeted against lipopolysaccharide-expressing microorganisms”]:

To determine (a) in parameter b) any existing cross-reactivity will always be taken into account. The calculation is as follows:
Having successfully established the value of n according to 2.): determine the number of antibodies (z) of n that are targeted against lipopolysaccharide-expressing microorganisms.

If (z)≧2 any existing cross-reactivity must be taken into account:
a. For evidence of cross-reactivity, a first column (or batch) is prepared with all except one of the lipopolysaccharide-expressing microorganisms (antigens) against which the (z) antibodies are targeted, with the antigens in excess relative to the total antibody content of the antibody product. A second column (or batch) is prepared with the one remaining lipopolysaccharide-expressing microorganism (antigen).
b. A sample of the antibody product is passed over the first and second columns in succession. The conditions ensure that, generally speaking, only specific antibody binding takes place. Each bound antibody component as a % by weight of the total antibody content of the antibody product is determined on the second column.
c. The process is carried out with all combinations of lipopolysaccharide-expressing microorganisms (antigens) against which the (z) antibodies are targeted.
d. If in all the processes an antibody component of ≧0.5% of the total antibody component of the antibody product is bound in the second column, then. z=(a).
e. If in one or more processes each of the antibody components that is bound to the lipopolysaccharide-expressing microorganism of the second pillar is <0.5% by weight of the total amount of antibody content of the antibody product, then cross-reactivity is occurring and should be taken into account in the determination of n in characteristic b). The lipopolysaccharide-expressing microorganism (antigen) with the lowest antibody component and a proportion of <0.5% of the total antibody content of the antibody product bound to the second column is not considered for the number (z): (z) is reduced by a value of 1 (=z−1).
f. This step should be repeated with a first column (or batch) prepared with all except one of the lipopolysaccharide-expressing microorganisms (antigens) against which the (z−1) antibodies are targeted, and each antigen should be in excess relative to the total antibody content of the antibody product. Thus the first column is not loaded with the lipopolysaccharide-expressing microorganism (antigen) that, in the first process, bound <0.5% by weight of antibodies and the lowest proportion of antibodies as a total of the antibody content of the antibody product, nor is it prepared with any other lipopolysaccharide-expressing microorganism (antigen) against which the (z−1) antibodies are prepared. A second column (or batch) is prepared with the one remaining lipopolysaccharide-expressing microorganism (antigen).
   A sample of the antibody product is passed over the first and second columns in succession. The conditions ensure that, generally speaking, only specific antibody binding takes place. Each bound antibody component is determined as % by weight of the total antibody content of the antibody product.
Steps c, d, e and f can be repeated as often as required mutatis mutandis until (z) is no longer variable. Then (z) corresponds to (a).

It is preferable that the conditions are selected such that, generally speaking, only specific antibody binding takes place. Thus the conditions for each specific antibody must be adjusted routinely. The following conditions have proven to be useful:
Determination at a temperature of 37° C.
Reaction time of 30 minutes to 1 hour
use of a buffer solution similar to the sample solvent (preferably a 50 mMTris buffer containing 0.14 M NaCl
pH value between 7 and 8.
intensive washing of the columns with 20 mM phosphate buffer, pH 7.0 or TTBS (0.3 M NaCl, 20 mM Tris/Cl, pH 7.8, 0.1% (v/v) Tween-20 and 0.01% NaN₃)+NaCl adjusted to 5 mM in order to remove non-specifically bound antibodies prior to elution.

If in doubt, these are the conditions that constitute the “conditions for specific binding in the determination of parameters (see above), specifically taking account of cross-reactivity”. Further details in this regard (test procedure, etc.) should preferably be taken from the assay procedure of the “ChickenIgG ELISA Quantification Kit” from Bethyl Laboratories, Inc. Of course, each antigen must match. If in doubt, the antigen is a complete microorganism so that a specific antibody is intended to include all the antibodies which bind to one of the epitopes of the microorganism after washing in the above-mentioned method.

A preferred embodiment of the invention relates to antibody products according to the invention, preferably according to the preceding embodiment, wherein one, several or all of the n-specific antibodies are polyclonal antibodies. Polyclonal antibodies are more economical and have a somewhat broader spectrum of efficacy than monoclonal antibodies.

A further preferred use of the invention, preferably according to one of the preceding embodiments, is characterized in that the n-specific antibodies are at least partially available in the form of monoclonal antibodies, polyclonal antibodies, primatized monoclonal antibodies, antibody fusion proteins, antibody fragments, conjugated antibodies, radioactively labelled antibodies, bispecific antibodies and/or monoclonal intrabody antibodies.

An equally preferred use of the invention, preferably according to one of the preceding embodiments, is characterized in that the n-specific antibodies are at least partially available in the form of monoclonal antibodies, where the monoclonal antibodies are selected from the group consisting of murine, chimeric, humanized and human monoclonal antibodies.

A particularly preferred use of the invention, preferably according to one of the preceding embodiments, is characterized in that the agent contains or is formed of immunoglobulin A, immunoglobulin D, immunoglobulin E, immunoglobulin M, immunoglobulin G and/or immunoglobulin Y.

Particularly preferred is an antibody product according to the invention, preferably in accordance with a preceding embodient, characterized in that it contains immunoglobulin Y (IgY).

A particularly preferred embodiment of the invention relates to antibody products according to the invention, preferably according to one of the preceding preferred embodiments, characterized in that the total proportion of n-specific antibodies is a maximum of 30% by weight of the total antibody content of the antibody product. Thus the efficacy spectrum for a range of clinical indications can be improved through non-specific antibody components.

An equally preferred embodiment of the invention relates to antibody products according to the invention, preferably according to one of the preceding preferred embodiments, characterized in that the total proportion of n-specific antibodies accounts for 2-90% by weight, preferably 10-65% by weight, most preferably 30-50% by weight of the total antibody content of the antibody product. The high antibody components mean that the dose can be reduced, thus alleviating the burden on the patient.

A further preferred inventive embodiment relates to antibody products according to the invention, preferably according to one of the preceding preferred embodiments, characterized in that a content of ≧50%, preferably ≧60%, preferably ≧70% by weight of the total component of n-specific antibodies is targeted against lipopolysaccharide-expressing microorganisms.

A particularly preferred embodiment of the invention relates to antibody products according to the invention, preferably in accordance with the preceding embodiments, characterized in that the antibody product is a drug or drug component and/or component of a prepared formulation.

In the context of the invention, drug or equivalent medication means substances or compound substances that are known to have properties useful for treating or preventing human or animal diseases or that are used in the human or animal body or a person, or that can be used in humans and animals, or that can be administered to humans and animals, either to restore, correct or influence human or animal physiological functions through pharmacological, immunological or metabolic effect, or for the purpose of establishing a medical diagnosis. In this text the word “drug” should preferably be understood as a substance and/or appropriate mixture of substances that can or must be the subject of a drug licence in the relevant country of application, particularly preferably a licence under German pharmaceutical law. The preferred drugs referred to in this application text also include so-called “orphan drugs” subject to simplified licensing procedures and that are preferably licensed under European and/or U.S. law.

A particularly preferred embodiment of the invention relates to antibody products according to the invention, preferably according to one of the preceding preferred embodiments, characterized in that the maximum value of n is 10, preferably the maximum value is 8 and more preferably the maximum value is 6. Thus the proportion of each specific antibody is high enough to ensure their efficacy for a variety of indications.

A particularly preferred embodiment of the invention relates to antibody products according to the invention, preferably according to one of the preceding preferred embodiments, characterized in that the n-specific antibodies act independently of each other to target the microorganisms selected from the group consisting of:

a) Gram-negative bacteria are preferably selected from the group consisting of Streptobacillus moniliformis, meningococcus, Chlamydophila, chlamydia, spirochetes, cyano-bacteria, species of Proteobacteria strain, especially Enterobacteriaceae (Escherichia coli, Salmonella, Shigella, Klebsiella, Proteus, Enterobacter), Pseudomonas bacteria, Legionella bacteria, Neisseria bacteria, rickettsia bacteria, Pasteurella multocida bacteria and species of the Bacteroidetes strain, and

b) Bacteria that cause food poisoning, and

c) inflammatory agents, and

d) optionally other microorganisms

Surprisingly, our own studies showed that particularly good results can be obtained with an embodiment of the antibody product of the invention, preferably according to one of the preceding embodiments, characterized in that among the n-specific antibodies there is at least one specific antibody against each of:
a) Clostridium perfringens (in particular type C),
b) F 18 Escherichia coli and
c) Salmonella (in particular S. typhimurium).

For this particularly effective antibody product according to the invention, it is preferable that n=3.

What follows relates to a more preferred embodiment of the antibody product according to the invention, preferably according to one of the preceding preferred embodiments, characterized in that the antibody product contains at least one specific antibody against each of:
a) Clostridium perfringens (in particular Type C),
b) F 18 Escherichia coli and
c) Salmonella (in particular S. typhimurium).

Surprisingly, our own studies further showed that particularly good results can be obtained with an embodiment of the antibody product of the invention, preferably according to one of the preceding embodiments, characterized in that among the n-specific antibodies there is at least one specific antibody against each of:

a) Candida albicans,

b) Porphyromonas gingivalis,

c) Streptococcus mutans,

d) Clostridium perfringens type C,

e) F 18 Escherichia coli and

f) Salmonella typhimurium.

For this particularly effective antibody product according to the invention, it is preferable that n=6.

What follows relates to a still more preferred embodiment of the antibody product according to the invention, preferably according to one of the preceding preferred embodiments, characterized in that the antibody product contains at least one specific antibody against each of:
a) Candida albicans,
b) Porphyromonas gingivalis,
c) Streptococcus mutans,
d) Clostridium perfringens type C,
e) F 18 Escherichia coli and
f) Salmonella typhimurium.

A particularly preferred embodiment relates to antibody products according to the invention, preferably according to one of the preceding preferred embodiments, characterized in that at least one, preferably two, more preferably several adjuvants for the vaccination of the hens should be used in the manufacture of the antibody product.

In the context of the invention, an adjuvant means a substance that is used with an antigen to produce an antibody product and in a non-specific way alters or increases the efficacy of the antigen for the production of an antibody product (in particular by increasing the immune response to the antigen) as compared to use of the antigen without this adjuvant. In the context of the invention, the preferred adjuvants are aluminium compounds, mineral oils with or without inactivated mycobacteria, and complete and incomplete Freund's adjuvant.

A particularly preferred embodiment relates to antibody product according to the invention, preferably according to one of the preceding preferred embodiments, characterized in that when the antibody product contains specific antibodies against Salmonella typhimurium and Escherichia coli:
a) the n-specific antibodies each account for an antibody component of at least 8/n %, preferably 9/n %, and still more preferred 10/n % 11/n % 12/n % and 14/n %, by weight respectively, based on the total antibody content of the antibody product and/or
b) the total amount of n-specific antibodies is ≧10% by weight, preferably ≧12%

by weight, more preferably ≧14% by weight, ≧15% by weight, ≧17% by weight respectively based on the total antibody content of the antibody product.

In principle all sources known to experts such as colostrum or the blood of mammals, in particular cattle or the eggs or blood of birds can be used as a source for the antibodies to be used according to the invention.

A preferred embodiment of the invention relates to an antibody product according to the invention, preferably according to one of the preceding preferred embodiments, characterized in that at least some of the n-specific antibodies are obtained from poultry.

A particularly preferred embodiment of the invention relates to antibody products according to the invention, preferably according to one of the preceding preferred embodiments, characterized in that at least some of the n-specific antibodies are obtained from hens.

Antibody products according to the invention made at least partially from poultry, and hens in particular, offer inter alia the advantage of a surprisingly high tolerance in humans and/or animals. In addition, the preferred form of the antibody product can be manufactured with a high degree of purity (high concentration of n-specific antibodies) so that in therapeutic use the actual doses (the n-specific antibodies plus other components) are kept relatively small. Thus there is no burden on the patient associated with taking the antibody product according to the invention. Moreover, antibodies extracted from poultry, and hens in particular, are economical to produce because of the correspondingly large animal populations.

Surprisingly, our own studies showed that particularly good results were obtained by using antibody products according to the invention when at least part of the component of n-specific antibodies were from hens. There was a surprisingly stark contrast in patient tolerance when compared with antibody products according to the invention made from mammals, in this case mainly cattle.

A preferred embodiment of the invention relates to an antibody product according to the invention, preferably according to one of the preceding preferred embodiments, characterized in that the antibody product, and particularly the n-specific antibody component, is at least partially obtained from solid egg yolk powder, preferably dried defatted egg yolk powder.

Defatted egg yolk powder can be obtained using standard methods (removal of fat from dried egg yolk powder), preferably using hexane. Once the fat has been removed, the defatted egg yolk powder is dried again.

A particularly preferred embodiment of the invention relates to antibody products according to the invention, preferably according to one of the preceding preferred embodiments, characterized in that the antibody product contains hexane.

However, some patients in the patient group treated with the antibody products according to the invention are allergic or intolerant to hexane or suffer other unwanted side effects when taking antibody products according to the invention containing hexane. The proportion of hexane contained in antibody products according to the invention should therefore preferably be limited.

A particularly preferred embodiment according to the invention relates to an antibody product according to the invention, preferably according to one of the preceding preferred embodiments, characterized in that the antibody product contains hexane, where hexane accounts for a maximum of 10 mg of hexane in 1 kg of antibody product, preferably 8 mg of hexane in 1 kg of antibody product, more preferably 5 mg of hexane in 1 kg of antibody product.

Hexane is required to remove the fat from the egg yolk powder (as shown above). However, fat removal may be carried out using other solvents or combinations of solvents such as dimethyl ether, acetone, ethanol and/or carbon dioxide, rather than hexane.

Hence a more preferred embodiment of the invention relates to an antibody product according to the invention, preferably according to one of the preceding preferred embodiments, characterized in that the antibody product contains ethanol and/or carbon dioxide.

A particularly preferred embodiment of the invention relates to an antibody product according to the invention, preferably according to one of the preceding preferred embodiments, characterized in that the antibody product contains ethanol and/or carbon dioxide and does not contain hexane.

A preferred embodiment of the invention relates to an antibody product according to the invention, preferably according to one of the preceding preferred embodiments, characterized in that the antibody product, and particularly the n-specific antibody component, is at least partially obtained from liquid and/or dried egg yolk.

Surprisingly, our own studies showed that the broader antibody spectrum from natural biological sources, in particular egg yolk, was highly effective. The origin of the agent to be used in accordance with the invention should be matched regularly against the substantive co-substances of the agent. Thus antibody products derived from egg yolk typically contain substances like lipoproteins such as HDL and LDL, and the water-soluble proteins of the yolk, the α-livetin (80 kDa), β-livetin (45 kDa) and/or γ-livetin (150 kDa), which also contain most of the enzymes found in eggs (Ternes, Acker and Scholtyssek, egg and egg products, 1994).

A preferred embodiment according to the invention relates to antibody products according to the invention, preferably according to one of the preceding preferred embodiments, characterized in that the antibody product, at least in part, and particularly the proportion of n-specific antibodies, at least in part, were obtained from defatted egg yolk powder, where the defatted egg yolk powder contains at least 15% by weight, preferably at least 30% by weight, more preferably at least 45% by weight protein, and a maximum of 35% by weight, preferably a maximum of 20% by weight, more preferably a maximum of 15% by weight fat, and at least 1% by weight, preferably at least 8% by weight, more preferably at least 20% by weight, most preferably at least 33% by weight carbohydrates.

A particularly preferred innovative embodiment relates to antibody products according to the invention, preferably according to one of the preceding preferred embodiments, characterized in that the antibody product is or contains defatted egg yolk powder, where the defatted egg yolk powder has a maximum moisture of <15%, preferably <10% and more preferably <5%.

A particularly preferred embodiment according to the invention relates to antibody products according to the invention, preferably according to one of the preceding preferred embodiments, characterized in that the antibody product is or contains defatted egg yolk powder, where the defatted egg yolk powder contains at least 15% by weight, preferably at least 30% by weight, more preferably at least 45% by weight protein, and a maximum of 35% by weight, preferably a maximum of 20% by weight, more preferably a maximum of 15% by weight fat, and at least 1% by weight , preferably at least 8% by weight, more preferably at least 20% by weight, most preferably at least 33% by weight carbohydrates.

A particularly preferred embodiment of the invention relates to antibody products according to the invention, preferably according to one of the preceding preferred embodiments, characterized in that the antibody product does not contain lactose.

A high number of patients, particularly those suffering from chronic pain and those with faulty mechanical barrier function of the mucous membranes of the digestive tract, are lactose-intolerant. Lactose can be found in colostrum and milk. Lactose can be removed using a separation process of products, but this method is very costly. Accordingly, it is preferable that the antibody products according to the invention contain no lactose.

A particularly preferred embodiment of the invention relates to antibody products according to the invention, preferably according to one of the preceding preferred embodiments, characterized in that the antibody product contains lactase.

An equally preferred embodiment of the invention relates to antibody products according to the invention, preferably according to one of the preceding preferred embodiments, characterized in that the antibody product contains oligosaccharides.

Oligosaccharides in an antibody product according to the invention have the advantage of increasing the stability of the antibodies contained therein.

However, in the patient group to be treated with antibody products according to the invention, there is, as with lactose, an increased incidence of oligosaccharide-intolerance.

Thus a more preferred embodiment of the invention relates to antibody products according to the invention, preferably according to one of the preceding preferred embodiments, characterized in that the antibody product contains less than 30% by weight, preferably less than 15% by weight, more preferably no oligosaccharides.

The advantage described above, which can be obtained by including oligosaccharides in antibody products according to the invention (to increase the stability of the antibodies), can also be obtained with innovative products containing sugar, where the sugar (e.g. trehalose or glucose) replaces the oligosaccharides.

A particularly preferred embodiment of the invention relates to antibody products according to the invention, preferably according to one of the preceding preferred embodiments, characterized in that the antibody product contains trehalose.

A particularly preferred embodiment of the invention relates to antibody products according to the invention, preferably according to one of the preceding preferred embodiments, characterized in that the antibody product contains trehalose and no oligosaccharides.

A particularly preferred embodiment of the invention relates to antibody products according to the invention, preferably according to one of the preceding preferred embodiments, characterized in that the antibody product contains at least one probiotic or substances derived therefrom.

Our own studies showed that there was a synergetic effect with regard to the efficacy of antibody products according to the invention if they contained at least one probiotic.

Probiotics in the context of the invention are defined as living microorganisms that enter the intestine in sufficient quantity and in an active form, with positive effects on health.

The main probiotic microorganisms used are strains of Bifidobacterium, Enterococcus, Lactobacillus, Lactococcus and Streptococcus. These living microorganisms are mainly effective in the digestive tract, where pathogens are inhibited or eliminated by the antibodies, particular the n-specific antibodies, contains in the antibody products according to the invention.

A particularly preferred embodiment of the invention relates to antibody products according to the invention, preferably according to one of the preceding preferred embodiments, characterized in that the antibody product contains yeast.

Our own studies showed that the presence of yeast in antibody products according to the invention had a positive impact and that treatment with yeast containing antibody products according to the invention was more effective. This additional efficacy is attributed to the positive effects of the yeast. The probiotic effect of live yeast cells in the intestine is seen as a positive effect of yeast. Yeast also contains nutrients and trace elements.

A particularly preferred embodiment of the invention relates to antibody products according to the invention, preferably according to one of the preceding preferred embodiments, characterized in that the antibody product contains lactase, yeast, ethanol and/or carbon dioxide, carbonic acid or carbonic acid salts.

A preferred embodiment of the invention, preferably according to one of the preceding embodiments, is characterized in that the agent forms part of a prepared formulation for administration, where the prepared formulation is selected from the group consisting of pharmaceutical preparations, cosmetic preparations, foodstuffs, food supplements, functional food and medical products, as well as animal feed, feed supplements and dietary supplements.

According to the invention, “functional foods” include foodstuffs and relevant newly developed products whose special ingredients means that they provide more than nutritional value and taste. Synonymous with these—although only partially, as they are different—re the terms Nutriceuticals, Foodsceuticals and Designer Foods, which are also embodiments of the preparation according to the invention.

The antibody-containing protein fraction of egg yolks can be pasteurized so that the product can be produced substantially free of pathogens without substantial loss of antibody activity. The starting material for the manufacture of a prepared formulation can be distinguished from yolk in foodstuffs by analysing the antibody spectrum, for example with ELISA or neutrality tests.

Usually, such raw materials (antibodies from egg products) undergo conventional concentration processes, such as common degreasing using solvents such as hexane, ethanol, acetone or carbon dioxide. Carbon dioxide may be preferred because of the residue-free finished product and the possibility that it eliminates the need for auxiliary materials such as oligosaccharides.

Other concentration processes include:
Hydroxy-Propyl-Methyl-Cellulose (Yokoyama H. et al., A 2-step procedure for purification of hen egg-yolk immunoglobulin-G-utilization of hydroxypropylmethylcellulose phthalate and synthetic affinity ligand gel, 1993, Poultry Science, 72, pp. 275-281.)
Polyethylene glycol, Dextran-Sulfate, Xanthan (Akita E. M., Nakai S., Comparison of four purification methods for the production of immunoglobulins from eggs laid by hens immunized with an enterotoxogenic E. coli strain, 1993, Journal of Immunological Methods, 160 (2), pp. 207-214.)
Ethanol (ToshioHorikoshi, et al., IgG Antibody from Hen Egg Yolks: Purification by Ethanol, 1993, Fractionation Journal of Food Science 58 (4), 739-742.)
Ultrafiltration (Hernandez-Campos F J et al., Purification of Egg Yolk Immunoglobulin (IgY) by Ultrafiltration: Effect of pH, Ionic Strength, and

Membrane Properties, Journal of Agricultural and Food Chemistry, 2009 December 8. [Epub ahead of print])
Lithium-Sulfate (Bizhanov G. et al., A innovative method, based on lithium sulfate precipitation for purification of chicken egg yolk immunoglobulin Y, applied to immunospecific antibodies against Sendai virus, 2004, Scandinavian Journal of Laboratory Animal Science, 31 (3), pp. 121-130.).

Accordingly, in addition to the agent used according to the invention, the mixture contains other co-factors that form the basis for the preparation to be used in accordance with the invention. These co-factors can have a positive effect on the action mechanism and/or tolerance of the preparation to be used in accordance with the invention.

A preferred embodiment of the invention relates to antibody products according to the invention according to the preceding preferred embodiment, wherein the prepared administration is selected from the group consisting of solutions, syrups, juices, tinctures, teas, extracts, percolates, powders, refined powders, granules, tablets, film coated tablets, soft gelatine capsules, hard gelatine capsules, oblongs, caplets, effervescent tablets, pills, suspensions, emulsions, pastes, creams, ointments, gels, lotions, suppositories, liniment, globules, buccal tablets, nanosuspensions, patches, transdermal patches, sprays, inhalants, and implants.

A preferred embodiment relates to antibody products according to the invention, preferably according to one of the preceding preferred embodiments, characterized in that the antibody product is a prepared formulation or part thereof, where the prepared formulation is in the form of tablets, and preferably in tablets with an enteric coating.

An enteric coating offers the advantage that the antibodies contained in the prepared formulation will not be denatured as they pass through the stomach.

A preferred inventive embodiment relates to antibody products according to the invention in accordance with the preceding preferred embodiment, characterized in that the formulation prepared for administration is available as tablets, and in that up to 50% of the tablets, based on the total number of tablets have an enteric coating.

A mixture of tablets with and without enteric coatings has the advantage that the uncoated tablets being to have an effect as soon as they are in the mouth. This is particularly advantageous for certain treatments with antibody products according to the invention.

Further preferred embodiment, the invention relates to antibody products according to the invention used in the preceding preferred embodiment, characterized in that the formulation prepared for administration is in the form of a mouthwash, where the mouthwash can be swallowed after a certain exposure time. Thus particularly preferred is a combination of antibody products according to the invention that are available as a prepared formulation in the form of enteric-coated tablets and/or capsules, and antibody products according to the invention that are available as a prepared formulation in the form of a mouthwash. (e.g. for mucositis).

A particularly preferred embodiment of the invention relates to antibody products according to the invention, preferably according to one of the preceding preferred embodiments, characterized in that the antibody is suitable for oral treatment.

Following oral ingestion, the antibody according to the invention binds to its specific antigens (e.g. toxins) as they pass through the digestive tract, and also attach to mucous membranes with defective barrier function, where they then give the antibody- or antigen-specific biological signal for apoptosis to the immune cells that have just absorbed endotoxins at the same site.

Oral (enteral) treatment has several advantages over parenteral administration forms (e.g. intravenous, intramuscular, subcutaneous): oral administration when used according to the invention is associated with significantly fewer side effects, because the agent (or antibodies and/or antibody parts) does not enter the bloodstream. It is digested like any other (food) protein as it passes through the gastrointestinal tract, before entering the body in the form of simple amino acids. Parenterally administered proteins (e.g. from blood plasma or serum), however, may be tolerated or rejected by the immune system. Only human plasma or serum proteins are tolerated by the parenteral route with an acceptable risk of side effects. Oral administration, however, relies on natural tolerance of proteins in the digestive tract and also allows for the use of xenogeneic antibodies, which are primarily advantageous from an economic standpoint. In addition, the oral administration is comfortable and convenient for the patient. Administration of the drug does not require any access (e.g. venous). Compared to other forms of administration, improved patient compliance and, by extension, increased efficacy is to be expected.

A particularly preferred embodiment according to the invention relates to antibody products according to the invention, preferably according to one of the preceding preferred embodiments, characterized in that the antibody product is a prepared formulation or a part thereof, and where the prepared formulation is administered in a daily dose of 0.1 g to 10.0 g, preferably 1.0 g to 8.0 g, more preferably 2.0 g to 7.0 g.

A particularly preferred embodiment according to the invention relates to antibody products according to the invention, preferably according to one of the preceding preferred embodiments, which can be administered as part of treatment in a daily dose of 0.1 g to 10.0 g, preferably 1.0 g to 8.0 g, more preferably 2.0 g to 7.0 g.

If using a formulation with a higher concentration of antibody parts, the medical expert should of course adjust the dose of the prepared formulation accordingly. Thus another possible preferred embodiment of the invention, preferably according to one of the preceding preferred embodiments, is one that can be administered as part of treatment in a daily dose of 0.1 g to 5.0 g, preferably 1.0 g to 2.0 g, more preferably 0.1 g to 0.8 g. The efficacy of a formulation can be increased by the use of enteric coatings and/or encapsulation. It is also possible to combine a more highly concentrated formulation with enteric coatings and/or encapsulation.

A strongly preferred use of the invention, preferably according to one of the preceding embodiments, is characterized in that the treatment or prophylaxis is administered in a daily dose of a prepared formulation with an enteric coating or encapsulation, from 0.1 g to 5.0 g, preferably 0.1 g to 2.0 g.

An enteric coating or enteric encapsulation offers the advantage that the antibodies contained in the prepared formulation will not be denatured as they pass through the stomach.

A particularly preferred innovative embodiment relates to antibody products according to the invention, preferably according to one of the preceding preferred embodiments, which can be administered as a daily treatment for at least 8 weeks, more preferably for at least 12 weeks.

A particularly preferred embodiment according to the invention relates to antibody products according to the invention, preferably according to one of the preceding preferred embodiments, characterized in that the antibody product is a prepared formulation or a part thereof, and where the prepared formulation is administered as a daily treatment for at least 4 weeks, preferably at least 8 weeks, most preferably at least 12 weeks.

A most preferred embodiment of the invention relates to antibody products according to the invention, preferably according to one of the preceding preferred embodiments, for use in long-term treatment.

A most preferred embodiment of the invention relates to antibody products according to the invention, preferably according to one of the preceding preferred embodiments, for use in the treatment or prophylaxis of a disease from the group consisting of:
chronic fatigue syndrome (CFS)
polyneuropathy, mononeuropathy, autonomic neuropathy, and small-fiber neuropathy, especially in autoimmune diseases, diabetes mellitus type I and II, diabetes type A, B, C, D, E, F, G, H, polyclonal gammopathy and/or kidney dysfunction
peripheral nerve compression syndromes (such as carpal tunnel syndrome), ulnar nerve entrapment (cubital tunnel syndrome (sulcus ulnaris), Morton's metatarsalgia, Bernhardt-Roth syndrome (meralgia paresthetica), thoracic outlet syndrome (TOS))
reactive arthritis, in particular infectious arthritis, non-infectious arthritis, juvenile idiopathic arthritis, rheumatoid arthritis
arthrosis other than osteoarthrosis, particularly osteoarthritis, primary arthrosis and secondary arthrosis
enthesopathies in collagenosis
epicondylitis humeri radialis
achillodynia
calcaneodynia and heel spur
periarthritis humero-scapularis
Tietze's Syndrome (sternoclavicular joint arthropathy)
arthropathy of the sacroiliac joint
myoarthropathy of the masticatory apparatus, temporomandibular dysfunction
cervical spine syndrome after deceleration trauma
colonic diverticulitis disease
cancer
eczema
asthma
interstitial cystitis (painful bladder syndrome)
food allergies
allergy to light, particularly polymorphic light eruption
post-herpetic neuralgia
mucositis, especially oral mucositis and/or mucositis after radiation therapy (radiation-induced mucositis) and/or mucositis after and/or chemotherapy
mucosal ulcers in Behcet's syndrome
mucosal erosions in pemphigus vulgaris
mucosal lesions in scleroderma
mucosal lesions in Sjogren's syndrome
migraine without aura
cardiovascular diseases
irritable bowel syndrome
ulcerative colitis
Crohn's disease
Graft-versus-host disease
fibromyalgia

A further innovative embodiment of the invention is a method for preparing an antibody product according to the invention according to one of the preceding embodiments, including the following steps:
a) Immunizing each of n groups of animals with only one microorganism species and/or part of the only one microorganism species, where in each of the n groups a different microorganism species and/or part of the different microorganism species is used and where at least 2 of the microorganism species and/or parts of the microorganism species are lipopolysaccharide-expressing microorganism species or are derived therefrom.
b) Obtaining an antibody-containing fraction from each of the n groups
c) Mixing the antibody fractions obtained
d) If necessary, concentrating the antibody content of the antibody-containing fractions and/or of the mixture of antibody-containing fractions.

A component of the invention is also an antibody product which can be or is produced in accordance with the production method according to the invention.

It is possible to obtain a particularly high titre of specific antibodies in that the populations (groups of animals) are immunised with only one antigen and afterwards the fractions are mixed to form the antibody product. This is because when the animals are immunised with more than one antibody the total titre of specific antibodies decreases because the immune system is further burdened.

Accordingly, an antibody product which can be produced according to the method according to the invention is preferred, wherein each specific antibody is contained in the antibody product at least in a proportion of 6/n % by weight (in relation to the total antibody proportion). As already described above, n is preferably ≦10, more preferably ≦8 and particularly preferably ≦6. In many cases, n is most particularly preferably 3.

An antibody product which is or can be produced by the method according to the invention and which has the specifications of the antibody product according to the invention described in greater detail above is particularly preferred.

The invention is described below in more detail using examples, although the invention is not limited to these examples.

Unless otherwise indicated, all quantities are weights.

Methods of Determination:

Methods of Determination for a Specific Antibody:

The determination of a specific antibody in an antibody product is carried out according to the following ELISA procedure:
ELISA reagents:
   a) 0.05 M carbonate buffer or coating buffer
      i. Dissolve 0.21 g NaHCO₃ in 50 ml of distilled water (DW₂), storable at 4° C. for up to 2 weeks
      ii. Dissolve 0.265 g of Na₂CO₃ in 50 ml DW2, storable at 4° C. for up to 2 weeks
      iii. Before use: adjust the NaHCO₃ solution to pH 9.6 by adding Na₂CO₃ solution (approx. 22 ml Na₂CO₃ for 50 ml NaHCO₃)
   b) washing solution or PBS-T: PBS containing 0.05% Tween 20 (v / v)
   c) blocking solution: PBS-T containing 5% low-fat dried milk.
   d) substrate solution
      i. Dissolve 14.19 g Na₂HPO₄ in 500 ml of DW₂, storable at room temperature for up to 1 year
      ii. Dissolve 10.5 g C₆H₈O₇.H₂O (citric acid) in 500 ml of DW₂, storable at 4° C. for up to 1 year
      iii. Before use: Mix 25 ml Na₂HPO₄ solution with 25 ml citric acid solution (the pH value should be greater than 4.5). Add 20 mg O-phenylenediamine dihydrochloride (OPD) wrapped with aluminium foil to protect it from light, mix well to dissolve OPD and add 0.02 mg H₂O₂ before use.
   e) Stopping solution
      i. Dilute 4 ml 36N H₂SO₄ solution with 44 ml DW₂ to obtain 3N H₂SO₄
      ii. Storable at 4° C. for over 1 year.
ELISA method:
1. Antigen coating: Dissolve antigen in coating buffer in a concentration of 5-10 μg/ml. Mix well and dispense 0.1 ml into each well of a 96-well ELISA plate. Leave the plate to stand for 18 h at 4° C.
2. Rinse three times with washing buffer.
3. Dispense 0.1 ml of the blocking buffer into each well, incubate at 37° C. for 1 hour.
4. Wash the plate 3 times with washing buffer. After washing, the ELISA plate can be used in the next step, or stored after drying at 37° C. for 1 h at 4° C. for up to one month.
5. In a 96-well U-bottom plate, carry out standard dilutions of the antibody sample using a PBS-T buffer. Transferring 0.1 ml of antibody dilution to the ELISA plate, starting with the highest dilutions.
6. Incubate at 37° C. for 1 h.
7. Wash the plate 6 times.
8. Dispense 0.1 ml of a second antibody solution (HRP-labelled anti-chicken antibodies diluted to appropriate concentration) into each well, incubate at 37° C. for 1 h.
9. Wash 6 times.
10. Dispense 0.1 ml of the substrate solution into each well; leave to stand at room temperature for 20 min.
11. Stop the reaction by adding 0.1 ml of the stopping solution. Determination at 490 nm with an optical density reader (OD-reader).

### EXAMPLES

Production of an Antibody Preparation According to the Invention:

According to the procedure in S. Hamada et al. (Infect Immun, 1991, 59: 4161-4167), populations of approximately 18-week-old LTZ or Hy-Line hens are immunized with an intramuscular injection of an emulsion mixture (approx. 1 ml) containing an antigen-type (see under “Production of antigens”) and an adjuvant. Several substances can be used as adjuvants. Possibilities include mineral-oil-based adjuvants, complete and incomplete Freund's adjuvants, or other adjuvants that increase the effect of the antigen. Vaccination is repeated every 6 to 15 weeks. The eggs of vaccinated hens can be collected over several weeks, broken open, the yolks separated, and then spray-dried, defatted (e.g. with hexane) in accordance with H. Suzuki et al, Aliment Pharm Ther, 2004; 20 (Suppl 1):185-92. Direct purification of the liquid egg yolk is also possible. This produces a product with a high concentration of IgY.

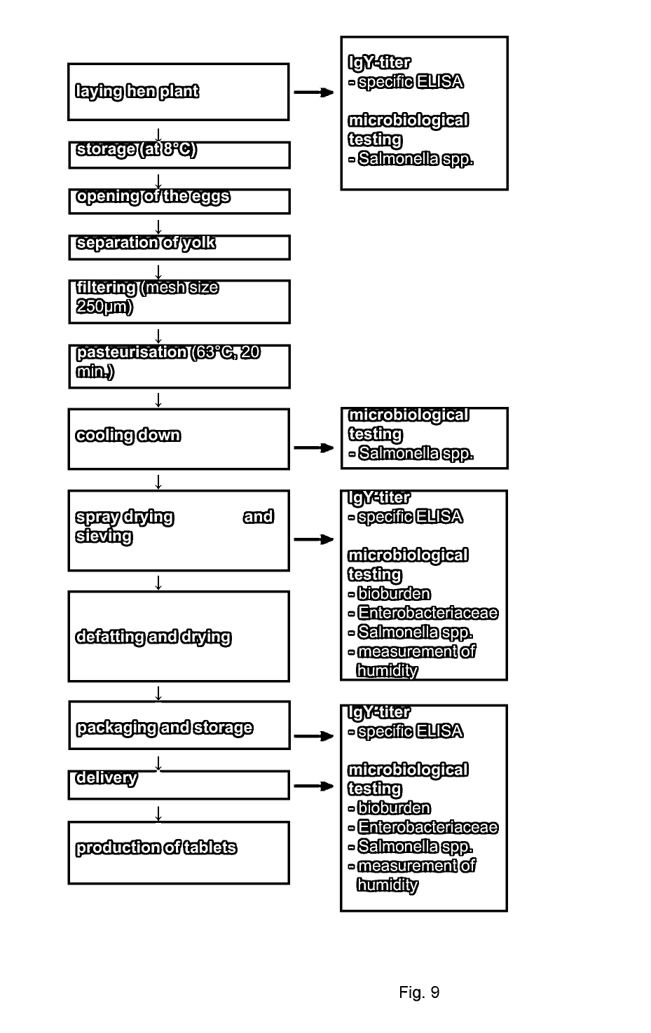

The preparation of an antibody preparation according to the invention is shown for illustrative purposes in FIG. 9 in the form of a flow chart. Here, before preparing the innovative product, the egg yolk (or purification products of egg yolk) from different populations immunized with different antigen types are mixed together.

A dried, defatted egg yolk powder mixture prepared according to the invention can be used for production of tablets: egg yolk powder mixture is combined for tableting with auxiliary materials such as isomalt, cellulose, silica, talcum, Kollidon, and/or magnesium stearate in an amount of 20% to 50%. The tablets produced contain approx. 10% by weight specific antibodies, based on the total antibody content of the tablet.

Since these are biologically produced preparations according to the invention, the medical expert should be aware that the proportions of the ingredients are subject to biological variations.

Provenance of the (particularly preferred) antigen types used:

For the production of the sample preparation 1 used in the examples below, the following antigens (=n antigens) were obtained:
from Streptococcus mutans: CA-GTA S. mutans Serotype C is extracted from the cells of MT8148 (as per Hamada et al, J Gen Microbiol, 1989, 135:335-44 and Infect Immun 1991, 59 (11):4161-4167).
from Poryphyromonas gingivalis: Using centrifugation/extraction the gingipain enzyme is obtained from the membrane of Poryphyromonas gingivalis (ATCC 33 277) (as per K. Yokoyama et al, Journal of Oral Science, Vol 49, No 3, 201-6, 2007).
Candida albicans:—cells (JCM 1542) are extracted by centrifuging them at 4° C. for 10 minutes at 8,000 rpm, using sterile phosphate buffered saline (pH 7.2) The cells are then resuspended in PBS and placed in an ultrasonic ice bath for 10 min. The sonicated cells were dialyzed against PBS. The protein concentration is determined by the BioRad protein assay system (BioRad Laboratories, CA, USA) (as per E M Ibrahim et al., Vaccine 2008, 26, 2073-2080).
Escherichia coli: whole cells of Escherichia coli F18, Serotype F107 (107/86), described by H. Yokoyama et al, The Journal of Veterinary Medical Science, Vol 59, No. 10, pp. 917-921, 1997.
from Clostridium perfringens: The alpha- and beta-toxins of Clostridium perfringens type C (NCTC3227) is obtained (as per M. W. Odentaal, Purification of the alpha toxin of Clostridium perfringens type A by ultrafiltration and gel chromatography). Onderstepoort J. Vet. Res., v. 54, p. 39-43, 1987 and J. Sakurai et al., Purification and characterization of Clostridium perfringens beta toxin. Toxicon Volume 25, Issue 12, 1987, Pages 1301-1310offenbart).
from Salmonella typhimurium: the antigen is obtained from Salmonella typhimurium cells (ATCC-13311) (as per H. Yokoyama et al., Vaccine, Vol 16, No. 4, pp. 388-393, 1998 and H. Yokoyama et al., American Journal of Veterinary Research, Vol. 59, No. 4, pp. 416-420, 1998).

Sample Preparation 1:

For production of the preparation used in the following examples the following steps were carried out:
populations of about 12 to 30-week-old LTZ or Hy-Line hens were immunized as indicated in the above-mentioned references with the applied antigen types. In the case of immunization with alpha- and beta-toxins from Clostridium perfringens type C (NCTC3227), immunization was carried out as per H. Yokoyama et al, The Journal of Veterinary Medical Science, Vol 59, No. 10, pp. 917-921, 1997:
Repetition of vaccination every 10 weeks and collection of the eggs of vaccinated hens over several weeks from week 2 following the second immunization.
Storage of the eggs at 8° C.
Breaking open of eggs
Separation of egg yolks; each egg yolk contains a specific antibody fraction of approx. 10% by weight based on the total amount of egg-yolk antibody content, targeted against the specific antigen with which the corresponding hen had been vaccinated.
Filtration of yolk with a 250-micron mesh
Mixing the yolks of the eggs to give an egg-yolk mixture arose containing almost equal proportions of specific (polyclonal) antibodies against the inserted antigen types; the egg yolk mixture contains a specific antibody content of 10% by weight, based on the total antibody content of the mixture
Addition of oligosaccharides (about from 2 to 25% based on the total amount of egg yolk powder e.g. ISO of NissiCo, Ltd. Nagoya, Japan) to ensure further processability after fat removal.
Pasteurization of egg yolk mixture for 20 minutes at from 60 to 63° C. and cooling of pasteurized mixture
Spray-drying of pasteurized mixture to give egg yolk powder (Ternes et al (eds). Eggs and Egg Products, 1994, Parey, Berlin and Hamburg)
Sieving of the spray-dried egg yolk powder for homogenization and removal of lumps using a mesh size of 2-3 mm
Removal of fat from egg yolk powder, according to H. Suzuki et al, Aliment Pharm Ther, 2004, 20 (Suppl. 1):185-92
Drying of egg yolk powder after fat removal.

Egg yolk powder prepared in this way is used in the following examples and is referred to as the “preparation”. The preparation contained a total antibody content of about 2% by weight, based on the total weight of the preparation. The total antibody fraction contained about 10% specific antibodies against the antigen-types used, based on the total antibody content of the preparation. Each specific antibody type (directed against the antigen-types used) accounted for 10/6% by weight, based on the total antibody content of the preparation. Furthermore, in the preparation more typical egg yolk components were present (Ternes et al (eds). Eggs and Egg Products, 1994, Parey, Berlin and Hamburg). The fat content was approx. 5% by weight, total protein content approx. 55% by weight, carbohydrates approx. 27% by weight, ash approx. 3.5% by weight, and residual moisture of the powder used approx. 4% by weight.

Sample Preparation 2:

The sample preparation 2 was produced similarly to the sample preparation 1, but the following antigens were used for immunisation:
Eschericia coli F18 cells, serotype F107 (107/86),
alpha and beta toxin of Clostridium perfringens Type C (NCTC3227),
antigen according to H. Yokoyama et al. of Salmonella typhimurium cell (ATCC-13311).

The sample preparation 2 was further used in formulations such as effervescent powder and tablets (in particular enteric-coated tablets). The quantity of sample preparation 2 used is 0.375 g per tablet or 5 g per packaging unit of effervescent powder.

It should be emphasized once again that the decisive factor in the use and efficacy of the products according to the invention is the proportion of specific antibodies (polyclonal or monoclonal). Since the preparation used in the following examples is a product that was obtained from natural sources (hen's eggs), some variations in the ingredients may naturally occur and are unavoidable.

### Operating Example

Neutralisation Capacity of the IgY Preparation (Sample Preparation 2)

Blood was taken from a healthy test subject and was subsequently treated with heparin so that it no longer coagulates. Heparin blood is the result. The heparin blood was subsequently separated into its components in order to obtain the blood plasma. 2 ml of the blood plasma obtained in that manner were incubated with 2 ml of E. coli Control Standand Endotoxin (50 EU/ml) at 37° C. and 5% of CO₂ for 24 hours. The result is a basic plasma solution which contains a defined quantity of a standardized endotoxin. Additional indications relating to the standard endotoxin used may be taken from the description relating to Limulus Amebocyte Lysate, Endosafe Endochrome-K test system (U.S. License No. 1197).

Preparation of the Test Solutions:

Test Solution B: Test Solution According to the Invention Versus E. coli, Salmonella and C. perfringens (Sample Preparation 2)

In a step a), 100 μl were removed from the basic plasma solution produced and subsequently, in a step b), mixed homogeneously with 100 μl of the dissolved test substance (IgY; 0.25 g/ml, from sample preparation 2 dissolved in water). The dissolved test substance contained an admixture of antibodies according to the invention against E. coli, Salmonella and C. perfringens (sample preparation 2). In a subsequent step c), the solution produced in that manner was incubated at 37° C. and 5% CO₂ for an additional 3 hours. In a subsequent step d), the solution was diluted by adding water to an endotoxin concentration of 2 EU/ml taking into consideration the subsequent addition of the LAL reagent and, in a step e), incubated at 75° C. in a water bath for 5 minutes (inactivation).

The test solution inactivated in this manner was subsequently tested by means of the LAL test (Endosafe Endochrome-K test system). To that end, the inactivated test solution was mixed with the LAL reagent taking into consideration the method provided by the manufacturer and subsequently pipetted onto a 96-well plate. The testing was carried out by an ELISA reader and subsequent evaluation.

Test Solution A: Comparison Solution (Control Solution) Which Does Not Comprise Any Antibodies

Test solution A was produced similarly to test solution B with the difference that in step a) 200 μl of basic plasma solution were removed and step b) was not carried out.

Test Solution C: Comparison Solution Comprising Antibodies Not in Accordance with the Invention (IgG, Lactobin N; Dr. Wolz Zell GmbH)

Test solution C was produced similarly to test solution B with the difference that in step b) 100 μl of a Lactobin N solution (manufacturer: Dr. Wolz Zell GmbH) were used. The antibody concentration corresponded to the concentration in test solution B.

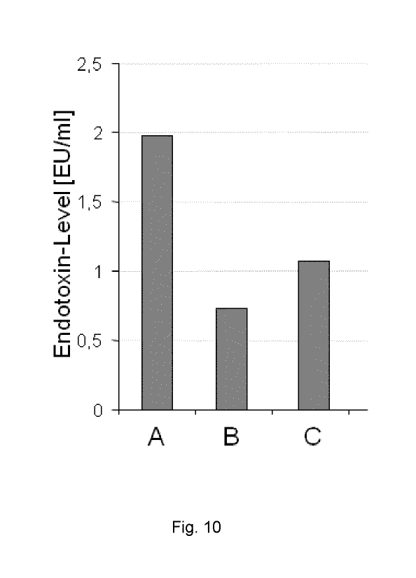

FIG. 10 (FIG. 10) shows the result of the ELISA test for the test solutions A, B and C. The test quantifies the quantity of free endotoxin in the test solutions, respectively.

Test solution A (control solution) which does not contain any antibodies in order to neutralise the endotoxins has the highest quantity of free endotoxin.

However, the test solution according to the invention has the lowest quantity.

In comparison with the test solution C (comparison solution with Lactobin N), the test solution B according to the invention has a substantially better neutralization of the endotoxin than using Lactobin N.

### Application Examples

### Example 1

Patient information: 32 years old, female

Duration of pain syndrome: 9 years

Diagnosis:

Complex regional pain syndrome (CRPS type II) after complex injury of the right thumb with fractures of the proximal and distal phalanges and nerve damage.

After 3 surgical operations (osteosynthesis of the fracture, removal of metal, surgical correction of deformed extensor tendons), there was persistent pain when at rest which increased during and especially after exercise (painful post-traumatic mononeuropathy).

Local Findings:

Burning, stabbing sensations in the whole of the thumb area, more acute at the site of the scars, with spontaneous shooting pain and pain triggered by gentle touch (allodynia), and referred pain when pressure applied (Tinel's sign on the damaged skin nerve). In addition, constant throbbing and stinging pain (deep pain) in the two proximal joints of the thumb. Radiographic evidence of arthrosis (pain consistent with osteoarthritis). Compared to the left thumb, the right thumb is significantly narrower, i.e. the skin and soft tissue of the thumb are thinner (atrophy).

Therapeutical Response of Pain:

No analgesic effect from central and peripheral analgesics, antidepressants, anticonvulsants, transcutaneous electrical nerve stimulation.

Opiate injections in the cervical sympathetic trunk of the sympathetic nervous system (GLOA) completely eliminates all pain for an average of 48 hours.

Evidence of a Significant Improvement in Symptoms with Oral Therapy of a Hyper-Immunoglobulin Against Endotoxins (LPS) from Immunized Hen Egg Yolks (Anti-LPS Hyper-IgY):

The patient participated in a treatment trial with anti-LPS hyper-IgY.The study period was 4 weeks, divided into two equal periods of 14 days with varying doses of the study preparation, testing of clinical efficacy (using a journal to document pain and quality of life indicators), and the effects on a broad spectrum of immunological laboratory parameters prior to and at the end of administration of the study medication.

Therapeutic Effect:

In the first two weeks of the study, daily intake of 2×1.25 g of the sample preparation (daily dose 2.5 g), including continued elimination of the deep throbbing, pounding pain in the structures near the joint of the right thumb (arthritic pain); neuropathic surface pain in the scar region was unchanged at this dose.

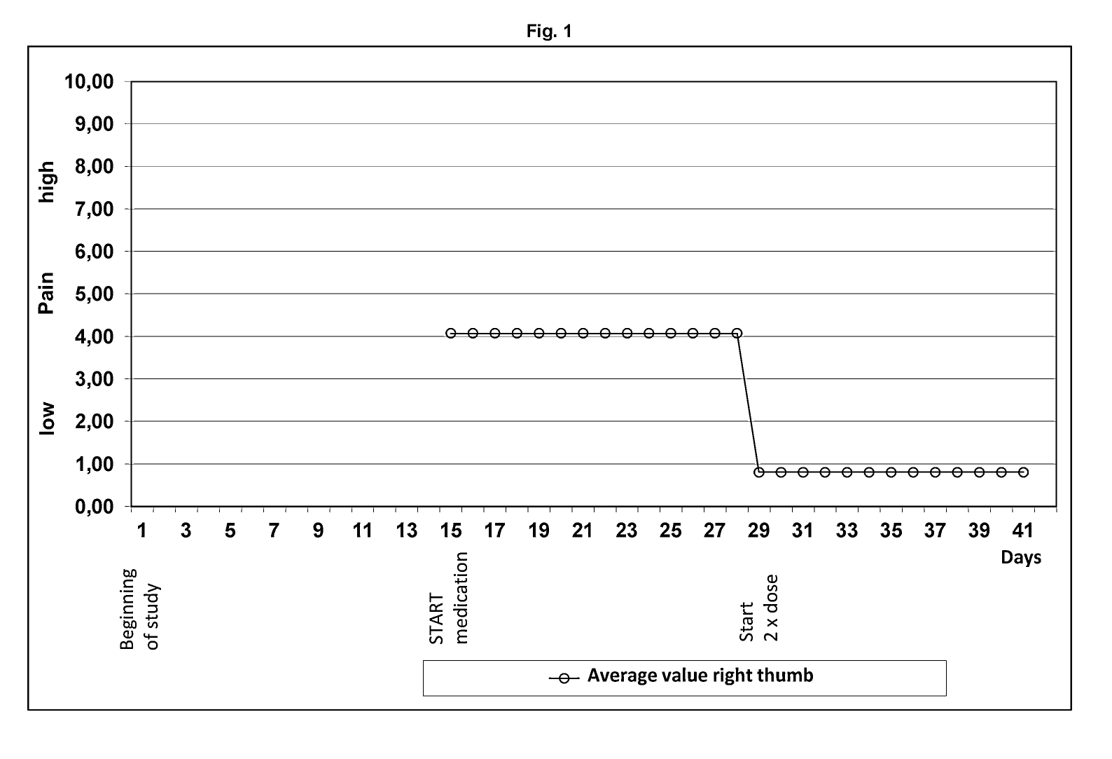

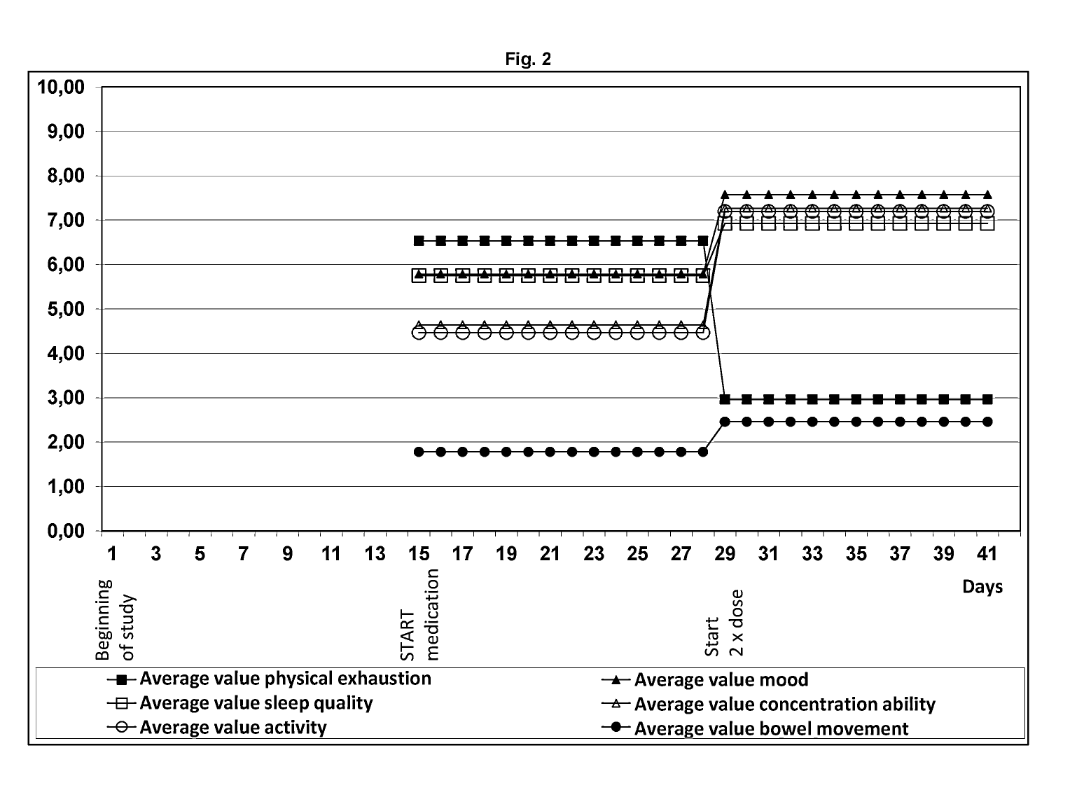

In the second study period, also over a period of two weeks, daily intake of 2×2.5 g of the preparation (daily dose 5 g), resulting in substantial improvement in neuropathic pain components (see FIG. 1).With significant recovery and freedom from pain in most hand functions, there was an improvement in concentration, range of activity, symptomatic daytime fatigue (chronic fatigue syndrome, physical exhaustion), sleep quality and mood (see FIG. 2).

In the laboratory part of the study, there was a significant reduction of endotoxin-activated monocytes in the peripheral blood (reduction through apoptosis), a decrease in the total number of monocytes to normal values; quantitative analysis of 22 immuno-messengers (chemokines, cytokines, growth factors) showed a variable but in principle consistent reduction in the plasma concentrations of inflammatory proteins and a significant increase in most anti-inflammatory protein factors.

Withdrawal Study:

After completion of the study the trial medication was continued due to a lack of alternatives. Treatment with IgY medication was withdrawn twice, and pain and general symptoms returned within 4-5 days.

Summary:

Neuropathic pains in the injured peripheral nerves have previously been presented as independent diagnoses of known aetiology and pathogenesis and these characteristics differentiated such pains from idiopathic pain syndromes. Some drugs are approved for the treatment of painful mono-and polyneuropathies, and there are evidence-based treatment recommendations that include non-drug options. However, a gross mismatch between the quality of therapeutic effects and the extent of side effects and complications points to significant gaps in patient care and the scientific basis.

The surprisingly positive effect of the specific IgY preparation in this female patient indicates that blood cell-borne endotoxins can, in individual cases, play a causal role in the chronification of pain, even in an injury-induced mononeuropathy.

FIG. 1 is a graphical representation of the pain level as the mean value of the numerical ratings for the two study periods.

The ordinate shows the numerical rating scale (NRS).The ordinate has a value ranging from 0 to 10, where 0 is no pain and 10 is the maximum pain imaginable. The abscissa gives the time in days.

Along with the visual analogue scale (VAS), NRS is the most common method of measuring acute and chronic pain so that meaningful conclusions about therapeutic effects can be made based on pain diaries. Patients with chronic pain are familiar with this method of assessing the intensity of their pain.

This patient began keeping the pain diary on starting the trial medication on day 15.

Instead of daily values, the mean values for both periods are shown.

FIG. 2 is a graphical representation of quality of life indicators (physical fatigue, concentration, mood, activity, quality of sleep, bowel movement).

The positive effects on quality of life indicators are particularly significant in terms of reduced daytime fatigue and increased range of activity. The numerical rating scale, where 0=no disease symptoms and 10=maximum disease symptoms, is similar to that used for assessing pain. Patients with chronic pain are familiar with the method of assessing the severity of their symptoms by keeping a pain diary.

### Example 2

Patient information: 39 years old, female

Duration of complex pain syndrome: Soft tissue rheumatism 30 years, arthrosis 20 years, neuropathy following nerve injury 5 years

Diagnoses:

1. Soft-tissue rheumatism

2. severe arthrosis with pain in the right knee joint on resting and exercising. Condition after joint replacements in both hips (arthrosis).

3. painful mononeuropathy of the left sural nerve with spasticity after irreversible nerve damage.

Local Findings:

Significant swelling and hyperthermia of the right knee joint with intolerable nocturnal resting pain, despite the use of ice packs and anti-inflammatories.

Right spastic equinus with pasty swelling, superficial burning sensation and shooting neuralgia. Tender musculature that fatigues easily, tenderness in tendon attachments and soft tissues of most joints.

Therapeutical Response of Pain:

Despite long-term treatment with antidepressants, anti-convulsives and anti-rheumatics, and the use of walking frames, the most important daily functions were severely restricted, mainly by pain.

Evidence of a Significant Improvement in Symptoms with Oral Therapy of a Hyper-Immunoglobulin Against Endotoxins (LPS) from Immunized Hen Egg Yolks (Anti-LPS Hyper-IgY):

Participation in the same study as the patient in example 1.

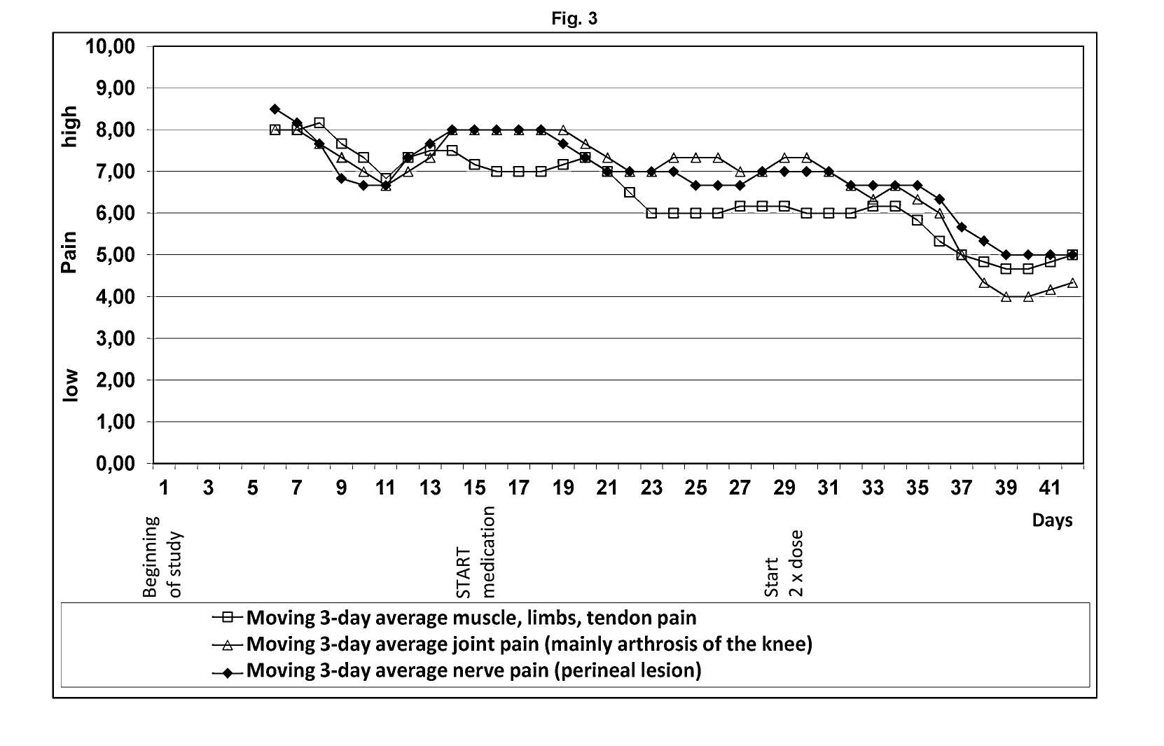

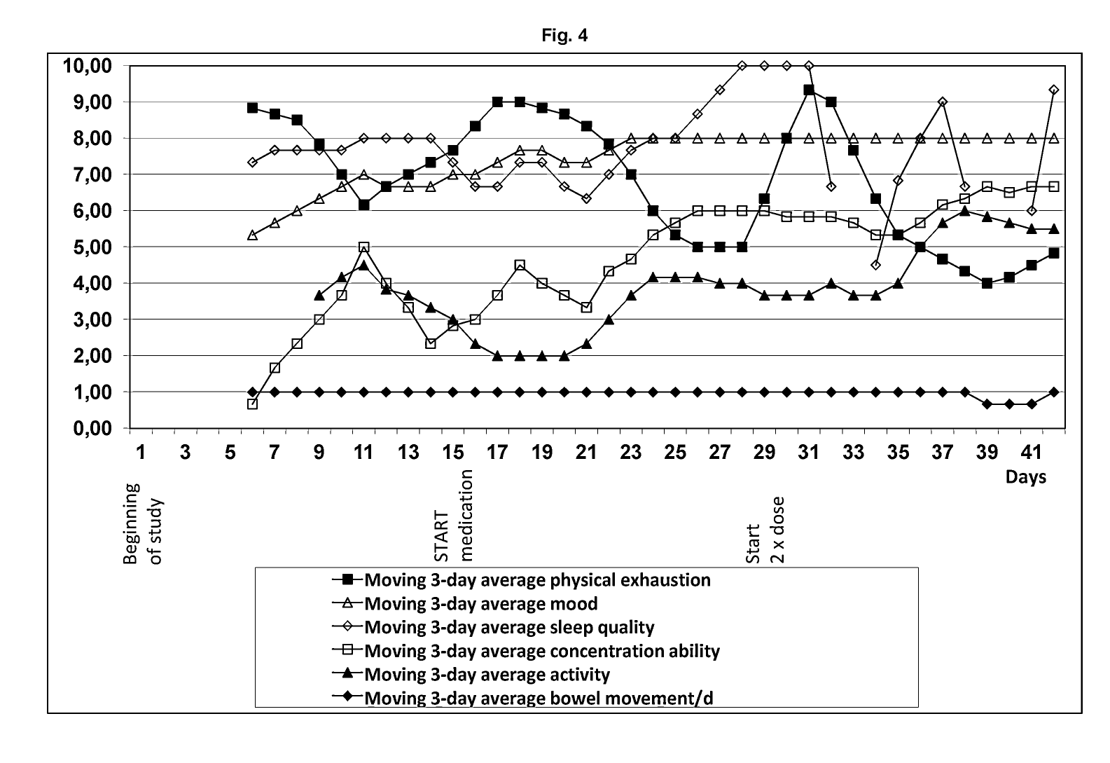

After 6 days of IgY treatment with the initial dose of 2×1.25 g, soft-tissue rheumatism was alleviated and there was an improvement in some of the general quality of life indicators (see FIG. 4). In the last 5 days of the study, a double daily dose (5.0 g in total) again brought about a significant improvement in all pain symptoms, which unexpectedly was most clearly apparent in the neuralgia of the left foot and the resting pain in the right knee joint. There was also a decrease in swelling and hyperthermia, even when anti-inflammatories were not taken (see FIG. 3).

In the laboratory part of the study, there was a significant reduction of endotoxin-activated monocytes in the peripheral blood (reduction through apoptosis), a decrease in the total number of monocytes to normal values; quantitative analysis of 22 immuno-messengers (chemokines, cytokines, growth factors) showed a consistent reduction in the plasma concentrations of inflammatory proteins and a significant increase in anti-inflammatory central protein factors.

Withdrawal Study:

Treatment with the sample preparation was continued, as the positive results could not be achieved by any alternative means. Efficacy was controlled and confirmed by 2 withdrawal studies carried out in the course of one year.

Summary:

Oral IgY medication was surprisingly effective on neuropathic pains associated with a mononeuropathy following peripheral nerve damage.

Moreover, in this case, the pain, swelling, inflammation and clinical signs of osteoarthritis in the knee joint improved subjectively and objectively.

Osteoarthritis is an independent diagnosis with objective radiological and clinical diagnostic criteria, as well as a very clear aetiology and pathogenesis.

The present example provides individual evidence that the endotoxin load of immune cells circulating in the peripheral blood is significantly reduced by orally administered antibodies, and that there is a causal relationship between the endotoxin load of the blood cells and the inflammation of osteoarthritis of the knee.

For endotoxins at least, such a causal relationship has never been investigated or proven and is therefore unexpected.

FIG. 3 is a graphical representation of the pain level of 3 pain phenotypes with a moving three-day average. The pain level is determined by the patient in a pain diary using the NRS method. Each measurement point is the mean value for the 3 preceding days (the “moving average”).

FIG. 3 shows that treatment with an anti-LPS-Hyper-IgYa triggers a synchronous reaction
of soft-tissue rheumatic pain (muscle, limb and tendon pain)
of pains in activated gonarthosis on the left (joint pain; mainly arthrosis of left knee)
of neuropathic pains after peripheral nerve damage (right nervus peroneus; neuralgia (perineal lesion)

FIG. 4 is a graphical representation of quality of life indicators (physical fatigue, concentration, mood, activity, quality of sleep, bowel movement) for the patient while receiving the trial medication, using a moving three-day average.

FIG. 4 shows that treatment with an anti-LPS-Hyper-IgYa results in a synchronous improvement in:
daytime fatigue (physical exhaustion)
concentration
range of activity
mental health (mood).

The severity of disease symptoms is represented by NRS on the ordinate. The measurement points on the ordinate are the mean values of NRS ratings from the patient diary for the preceding 3 days.

### Example 3

Patient information: 56 years old, female

Duration of complex pain syndrome: 13 years

Diagnoses:

1. complex regional pain syndrome in both upper extremities

2. Bilateral meralgia paresthetica=compression syndrome in skin nerves on the outer thigh where the nerves pass under the inguinal ligament.

3. Morton's metatarsalgia in both feet (compression syndrome in the nerves of the sole of the foot between the first and second toes)

4. Irritable bowel syndrome with severe bursts of abdominal pain (colic) predominantly localized in the lower abdomen

5. Painful bladder syndrome/interstitial cystitis (PBS/IC), a bladder disease of unknown aetiology. There is persistent pain in the bladder with a strong urge to urinate even when the bladder contains very little urine. It can be very painful to urinate. There was no evidence of any urinary tract infection or other localized pathology. Endoscopic examination of the bladder mucosa revealed signs of inflammation. The biopsy usually shows an increase in eosinophil granulocyte mastocyte inflammatory cells, which suggests an allergic inflammation.

6. Eczema

History and Local Findings:

Following a minor accident, the patient developed a complex regional pain syndrome (CPRS) in the left hand, which spread to the whole of the upper left extremity (shoulder-arm-hand syndrome). This is a combination of the three specific diagnoses: periarthropathy of the shoulder joint, epicondylitis humeri radialis, and CRPS of the hand. The patient also had nerve compression syndrome in the bony groove of the ulnar nerve at the elbow with loss of sensory and motor nerve function in the affected hand.

As the disease progressed, the patient also developed compression syndrome in the right metacarpal nerve (carpal tunnel syndrome). Following surgical treatment, complex regional pain syndrome developed in the right hand so that in addition to the pain, the patient suffered a complete loss of function of both hands.

Therapeutical Response of Pain:

While undergoing an unsuccessful daily interdisciplinary pain treatment over the course of 6 months, the patient developed a further compression syndrome of the peripheral nerves in the thighs and feet (meralgia paresthetica and Morton's metatarsalgia).

The full clinical picture, including eczema, was eventually treated successfully with intravenous administration of the human C1 esterase inhibitor (C1-INH) Berinert® in combination with low-molecular-weight heparin. As the patient did not have a C1-INH deficiency, this was an off-label medication (a treatment outside of approved indications).The treatment had to be followed up at intervals of an average of 8 weeks.

Evidence of an Equivalent Elimination of Symptoms with Oral Therapy of a Hyper-Immunoglobulin Against Endotoxins (LPS) from Immunized Hen Egg Yolks (Anti-LPS Hyper-IgY):

The patient was only accepted into the IgY therapeutic study described in examples 1 and 2 once the effects of the last Berinert® injections had subsided and all of the above disease symptoms, as well as the eczema and severe headaches, had returned.

Therapeutic Effect:

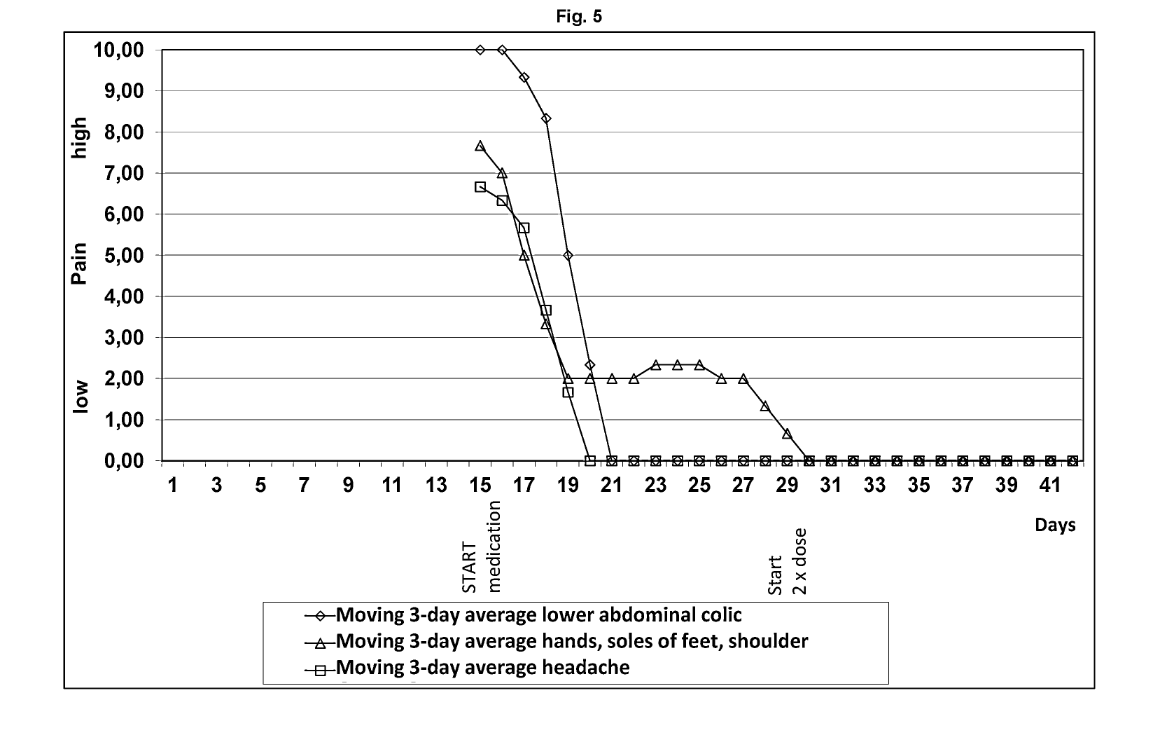

Within the first week of the study, the initial dose of 2×1.25 g IgY had completely eliminated headaches and abdominal pains. At the beginning of the second study phase (beginning of the third week of treatment with IgY), the neuropathic pains, sensory disturbances caused by nerve compression syndromes in both the lower and upper extremities and shoulder pain and restricted movement had all disappeared (see FIG. 5).The general disease symptoms of the complex health problem and the eczema disappeared along with the pain, although the daytime fatigue persisted at a low level to the end of the study period. On continuing treatment at the lowest initial dose, the symptoms of fatigue disappeared completely in the following months.

In the laboratory part of the study, there was a significant reduction of endotoxin-activated monocytes in the peripheral blood (reduction through apoptosis), a decrease in the total number of monocytes to normal values; quantitative analysis of 22 immuno-messengers (chemokines, cytokines, growth factors) revealed significant and successful changes brought about by the treatment, as with all study participants. In this patient, however, the greatest changes were in a different spectrum of chemokines.

Withdrawal Study:

Following the study, the sample medication was continued at a low dose over 4 months. Symptoms only returned when the study medication was stopped for a further 3 months.

Summary:

The effect on neuropathic pain was surprising, because the neuropathic functional disturbances was not caused by injuries to the peripheral nerves but rather by an almost generalized compression syndrome of the peripheral nerves. This effect occurred within one week at the lowest dose and was equivalent to Berinert® in terms of quality of treatment.

The pain and dysfunction in the shoulder and elbow joint caused by unusually acute periarthritis and epicondylitis were completely eliminated after 15-16 days. This very common form of inflammatory periarticular disease was indeed associated with the unknown systemic disease of the patient, but the surprisingly successful response to IgY therapy suggests that the general nature of this disease is an endotoxin-mediated disease of the musculoskeletal system. It is therefore likely that therapy with antibody preparations, particularly the specific IgY preparation, could be successful in treating an as yet unknown proportion of patients with these diseases.

By this analogy, the same can be assumed for irritable bowl and interstitial cystitis symptoms.

Even the patient's pronounced eczema did not return while she was receiving IgY therapy. Irritable bowel syndrome, interstitial cystitis and eczema have a common immunological feature: pathologically activated mast cells are involved in proven inflammatory organ changes. This cell type of the immune system also has binding sites for endotoxins, so that in particular circumstances these cells can be activated simultaneously in various organ systems by this toxin. In specific oral antibody therapy with IgY, endotoxins are partially eliminated in the gut.

FIG. 5 shows the change in intensity of 3 different classified pain symptoms (the three most acute pain phenotypes) of this patient through self-assessment using NRS values after initial treatment with the specific IgY preparation. The measurement points on the ordinate are the mean values of NRS ratings from the patient diary for the preceding 3 days. The abscissa gives the time in days. The patient began taking IgY on day 15 and the dose was doubled from day 29.

### Example 4

Patient information: 55 years old, female

Duration of complex pain syndrome: 12 years

Diagnoses:
1. Post-herpetic neuralgia of 1st and 3rd tight trigeminal nerve (trigeminal mononeuropathy after herpes zoster)
2. migraine without aura
3. bilateral achillodynia (inflammatory enthesopathy of the Achilles tendon) with signs of autoimmune disease (undifferentiated collagenosis)
4. Irritable bowel syndrome with episodic diarrhoea
5. Secondary antibody deficiency syndrome (IgG and IgA)
6. Chemical laboratory evidence of an autoimmune disease (autoantibodies) relating to an undifferentiated collagenosis

History and Local Findings:

A long time ago (>10 years) the patient had herpes zoster (shingles) on the forehead and upper jaw of the right trigeminal nerve. Once the viral infection had cleared, chronic pain, numbness and episode of acute pain persisted, especially behind the right eye, the nose and around the right edge of the tongue (post-herpetic neuralgia).

The facial pain attacks continued daily before or after an episode of diarrhoea, which was characterized by a rapidly occurring watery bowel movement (irritable bowel syndrome).

The facial pain attacks were also always accompanied by increased sweating and/or chills.

Prior to the facial neuralgia, there had been migraine without aura, which until the start of IgY therapy had lasted for an average of 7 days each month.

At a later stage in the disease, the patient began to suffer from a bilateral achillodynia, which gradually led to significant mobility problems. Achillodynia is a painful disease of the Achilles tendon, which occurs either as an independent inflammatory disease—for example by straining the tendon—or as a secondary symptom of a rheumatic disease.

Therapeutical Response of Pain:

Long-term analgesic medication consisted of a combination of 6 different drugs: An antiepileptic drug (Pregabalin), 2 antidepressants (amitriptyline and duloxetine) the analgesic Flupirtine and the opioids Tilidine and Tentanyl (200 μg stick if necessary). The patient had already consulted 9 specialist pain, neurological and orthopaedic clinics (for achillodynia). She suffered from all 3 pain syndromes without relief, as well as irritable bowel syndrome and the side effects of many medications.

Evidence of a Significant Alleviation of Symptoms with Oral Therapy of a Hyper-Immunoglobulin Against Endotoxins (LPS) from Immunized Hen Egg Yolks (Anti-LPS Hyper-IgY):

Treatment with the specific IgY was started at the lowest trial dose (2×1.25 g). Once treatment had begun the migraines disappeared. The achillodynia and the irritable bowel symptoms also disappeared within one month.

The post-herpetic neuralgia was unchanged at this dose, as was drug use. By doubling the dose (2×2.5 g) after 3 months of treatment at the lowest trial dose, the facial pain attacks became much less frequent and the duration of the attacks was shortened from one hour to just a few minutes. The intensity of the pain remained unchanged. Some medications were dispensed with completely, while others were continued at a reduced dose. The patient's general condition improved radically.

By substituting the (relatively minor) antibody deficiency with intravenous human immunoglobulins, all the remaining pain and disease symptoms disappeared completely, although numbness in the facial skin and tongue persisted.

Summary:

Post-Herpetic Neuralgia

Post-herpetic neuralgia is an independent clinical diagnosis of known aetiology. This is a mononeuropathy consisting of permanent nerve damage after a viral infection. Science has not come up with a satisfactory explanation as to why only some patients with shingles go on to develop post-herpetic neuralgia, which often remains untreatable for the rest of a patient's life. The binding of endotoxins to the nerve roots damaged by the infection could be one of several mechanisms that are a partial cause of persistent pain. The present case study strongly supports this hypothesis: While being treated with the specific IgY preparation, the patient's migraine, irritable bowel syndrome and inflammatory changes in the Achilles tendon disappeared. Such a significant impact can only be understood in terms of the neutralizing effect of IgY on the transport of endotoxins in the body. The significant reduction of the duration of facial pain episodes and their reduced frequency suggest that the same induction mechanism is involved in this pain syndrome.

Achillodynia

The complete elimination of pain and inflammation in the Achilles tendon in the context of the autoimmune disease is surprising, particularly since no treatment had previously afforded the patient any relief. It is well known that inflammatory enthesopathies (an umbrella term for all inflammatory diseases of the tendon and tendon attachment) are frequently resistant to treatment.

Migraines

The patient's migraine could not be treated adequately with seizure prophylaxis (beta-blockers) and the specific migraine drugs taken by the patient during an attack were not effective enough to enable her to continue to work on days when she had migraines. This resulted in an average of 7 days of absence from work each month. This example clearly shows that the transfer of endotoxins has a unique and surprising part to play in this condition.

### Example 5

Patient information: 65 years old, male, study number 17

Duration of pain syndrome: Headaches for 35 years, neuropathy of the right sciatic nerve for one year, right epicondylitis for 3 years.

Diagnoses:
1. Persistent symmetrical tension-type headache since a severe episode of “tickborne encephalitis” (TBE) in 1974 (Inflammation of the brain and meninges caused by a tick bite)
2. Epicondylitis humeri radialis and ulnaris right with substantial impairment of the entire right arm and severe resting pain.
3. Lumbar back pain radiating across the right sciatic nerves, the residual effect of surgical treatment for a herniated disc a year ago (mononeuropathy of the sciatic nerves after pressure injury).

Local Findings:

Very tender periosteum around the muscle attachments of right forearm muscles on the upper arm in the elbow area. Radiating pain during typical movements with tension in muscle attachments (turning screws, writing, holding objects with an outstretched arm, such hanging coats on door hooks).

Throbbing pain in the lumbar spine, pain in the sciatic nerve when passively raising the right leg in a stretched position while lying down (positive Laseque's sign), Achilles tendons and right patellar tendon reflex absent, no motor weakness in right leg.

Therapeutical Response of Pain:

Having taken medication for the meningo-encephalitis for many years with severe side effects (liver damage), the patient no longer takes any medication for his pain. Physiotherapy as part of an inpatient rehabilitation programme (after the meningo-encephalitis and the disc surgery) had no beneficial long-term effect.

Evidence of a Significant Improvement In, and In Some Cases Complete Elimination of, Symptoms with Oral Therapy of a Hyper-Immunoglobulin Against Endotoxins (LPS) from Immunized Hen Egg Yolks (Anti-LPS Hyper-IgY):

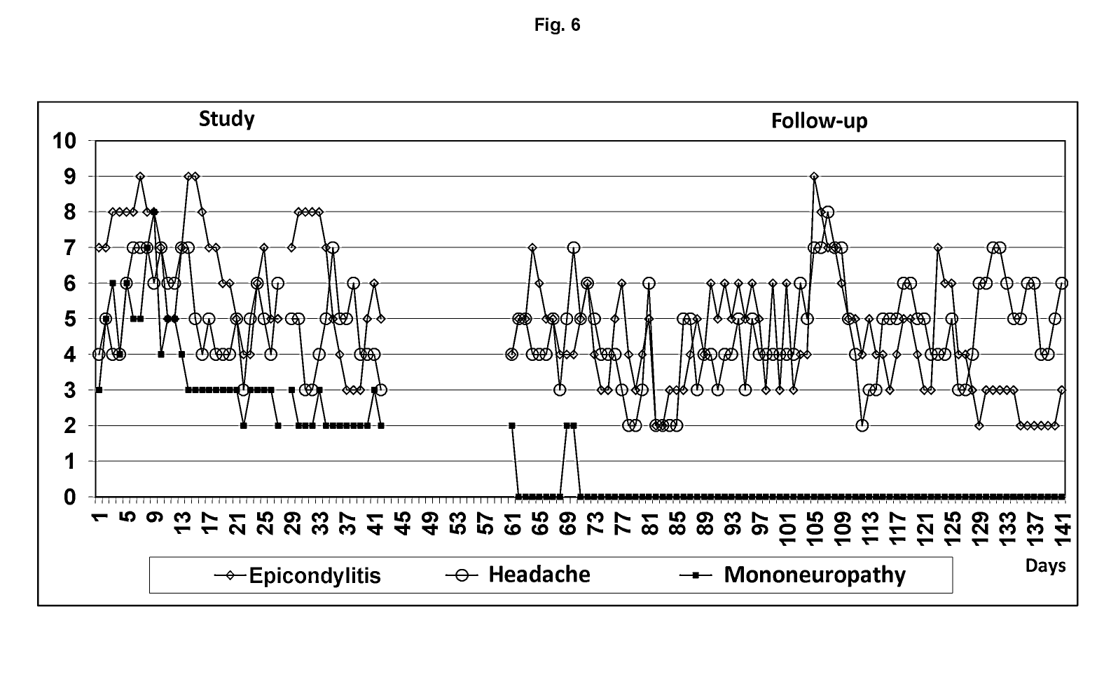

During the study there was an improvement in all 3 pain phenotypes, the significant variations in pain level recorded before the study remained, the mean values began to decrease during the low-dose phase (2×1.25 g daily), and this effect was even clearer at the higher dose (2×2.5 g daily). In the follow-up observation phase, the back and sciatic pain disappeared completely while the patient remained on the higher dose. The pain and functional disturbances caused by the epicondylitis were reduced so much that the patient no longer experienced any impairment, because the pain was low even under physical stress. During the follow-up phase, the headache only occurred early in the morning and it was no longer of significance to the patient (see FIG. 6). In the graphical representation of the numerical figures used to rate the pain level in the pain diary, the patient had not entered daily average values but rather the maximum values apply for each day, so that the patient's assessment—relayed verbally—that he was largely pain free is not reflected.

In the laboratory part of the study, there was a comparatively small reduction in endotoxin-activated monocytes in the peripheral blood (reduction through apoptosis), a slight decrease in the total number of monocytes to normal values; quantitative analysis of 22 immuno-messengers (chemokines, cytokines, growth factors) showed, in comparison to other study participants, a disproportionate reduction in the plasma concentrations of inflammatory proteins (e.g. TNFα, IL-6, IL-8), with the highest values for the increase in anti-inflammatory protein factors (e.g. interleukin 4 and 5).

Withdrawal Study:

The patient finished taking IgY after 141 days for a period of 6 months until the headaches and pain of the epicondylitis began to affect quality of life again. Thereafter he took the medication only as needed for periods of 4-5 days, and was able to control the pain level as desired.

Summary:

Chronic Headache After Meningitis, Mononeuropathy of Sciatic Nerves After Nerve Root Compression, Epicondylitis

The monocyte-bound “endotoxin load” in the patient's blood seems to be implicated in the aetiology of 3 chronic pain phenotypes that are normally diagnosed separately in medicine. The residual damage to the brain and meninges (TBE) following viral infection, and to the sciatic nerve following root compression are the biological weak points where endotoxin-laden immune cells become attached and prolong a local immune response (pain). The causal explanation for this epicondylitis place is the immune activation in the neural supply of the right arm (and not the painful elbow).

FIG. 6 illustrates the changes in intensity of 3 different classified pain symptoms (epicondylitis, headache and back-sciatic pain/mononeuropathy) in this patient based on self-assessment of NRS scores over the study period and during subsequent follow-up, when the patient continued to take IgY at a low maintenance dose until day 141.

The measurement points on the ordinate correspond to NRS values that were removed from the diary (there was no 3-day average due to lower fluctuations in pain levels). The abscissa gives the time in days. IgY treatment began on day 15 and continued in a double dose from day 29 to day 42. Thereafter the treatment was maintained at a low maintenance dose until day 141.

### Example 6

Patient information: 65 years old, male

Duration of pain syndrome: 11 years with interruption of symptoms for 3.5 years after operation (the Jannetta procedure).

Diagnoses:
1. Trigeminal neuralgia, II. and III. Right limb (diabetic mononeuropathy)
2. Type I diabetes with insulin pump, diabetic distal leg polyneuropathy, diabetic nephropathy (proteinuria, with normal function)

History and Local Findings:

The neuralgia began more than 10 years after onset of diabetes, first line treatment was carbamazepine, then other anticonvulsants, and then microvascular decompression of the trigeminal root (MVD or the Jannetta procedure). 3.5 years pain-free. When the attacks began again, the dose of carbamazepine was increased rapidly, leading to side effects affecting the central nervous system, with the threat of disability (threatened loss of driving license, professional driver).

Findings:

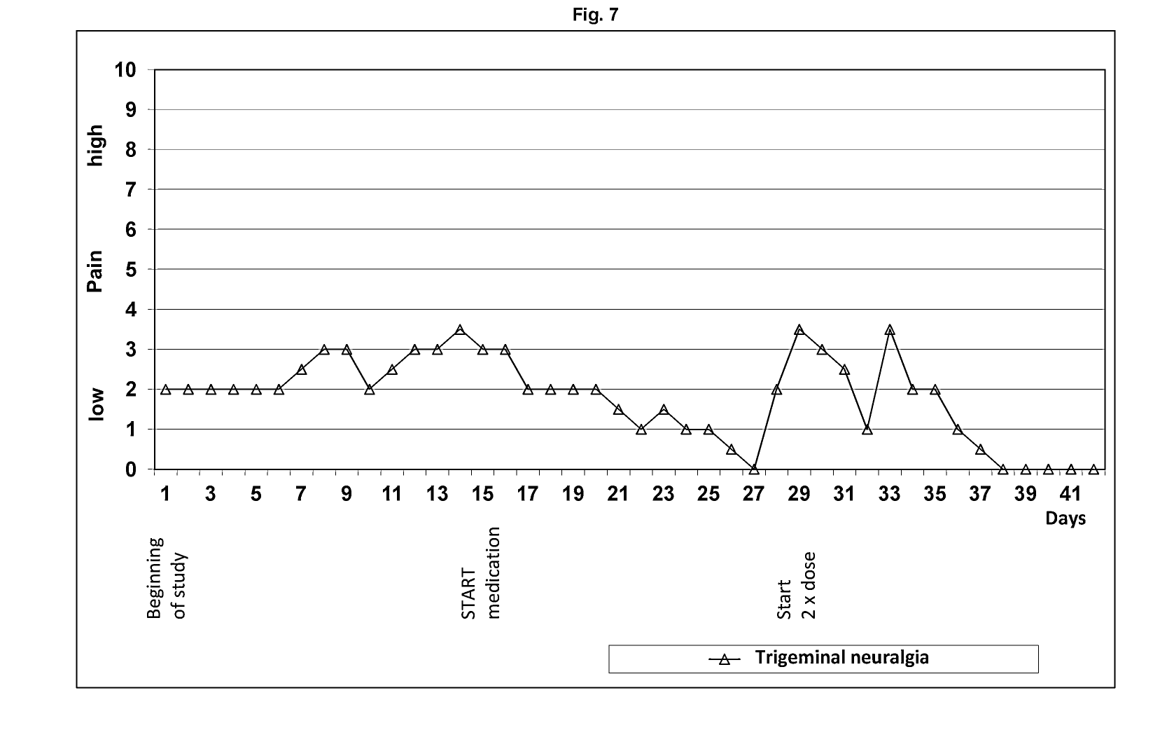

Tingling sensations in a small area of the upper lip and the oral mucosa of the cheek in the lower jaw. Strongly avoids creating air currents or disturbances that might trigger pain attacks in these areas. Repeated bursts of pain in quick succession brought on by eating, barely tolerable even with toxic levels of the antiepileptic drug in the blood (apparently low level of pain at start of study in FIG. 7). Work stress and personal issues increased the likelihood of an attack.

The distal leg polyneuropathy is sensory; symptoms include the sensation of walking on cotton wool, and oedemas in the feet and lower legs.

Evidence of Complete Remission (Elimination) of Trigeminal Neuralgia and Improvement of Symptoms of Diabetic Polyneuropathy with Treatment with a Hyper-Immunoglobulin Against Endotoxins (LPS) from the Egg Yolks of Immunized Hens (Anti-LPS-Hyper-IgY):

Within the first 14 days of study, at an IgY-dose of 2×1.25 g per day, the patient was pain-free, and the patient reduced the carbamazepine dose from 1200 mg to 900 mg with the effect that, while the side effects subsided, the attacks began to recur. On a double-dose of IgY 1.25 g 2 times daily, the patient was again pain-free, and carbamazepine was reduced to a daily dose of 450 mg for the remainder of the study (see FIG. 7). The sensory symptoms in skin and mucosal areas, which were the main sites of the pain attacks, disappeared completely during IgY therapy. For idiopathic trigeminal neuralgia, there are no sensory disturbances, with the exception of therapeutic interventions that cause damage to the nerves. This case was therefore a diabetic mononeuropathy in the area of the trigeminal nerve, and not an idiopathic form of neuralgia.

While on IgY-therapy, the patient recovered the sensation in both feet and in the lower legs and the tendency to oedemas in the feet and lower legs was significantly lower. In these regions of diabetic polyneuropathy, the patient had no pain. The effect on symptoms of polyneuropathy is surprising.

Thereafter, the patient, while taking a daily dose of IgY of 5 g, was able to stop taking the antiepileptic drug carbamazepine after one month; the patient remained symptom-free on an IgY maintenance dose of 2.5 g per day. On repeated attempts to lower this daily dose, the sensory disturbances returned in the same places that the patient typically associated with pain attacks.

In the laboratory part of the study, there was a significant reduction of endotoxin-activated monocytes in the peripheral blood (reduction through apoptosis), a significant decrease in the total number of monocytes to normal values; quantitative analysis of 22 immuno-messengers (plasma concentrations of chemokines, cytokines, growth factors) showed a significant reduction in growth factors IGF-1 and GMCSF and proinflammatory cytokines IL-8 and IL-7, and an increase in anti-inflammatory cytokines IL-4, IL-5 and IL-13.

Withdrawal Study:

The patient could only reduce the dose of study medication (IgY 5 g daily dose), a period without treatment was not possible. The specificity of the effect of the dominant set of antibodies against endotoxin was compared with a therapy involving a hyper-immune IgY preparation against antigens of periodontal pathogens.

Summary:

Diabetic Mononeuropathy of the Trigeminus Nerve

The sustained elimination of the symptoms of idiopathic trigeminal neuralgia through long-term treatment with oral immunoglobulins from bovine colostrum has been clear for some time, but not for trigeminal neuralgia on the basis of diabetic mononeuropathy, as in this example.

Diabetic Polyneuropathy

In this example, the concomitant therapeutic influence of the sensory and autonomic components of polyneuropathy (loss of sensation and lower-leg oedema) gave the first surprising indication of the efficacy of the preparation in diabetic polyneuropathy.

FIG. 7 illustrates changes in the intensity of pain attacks resulting from trigeminal neuralgia by means of patient self-assessment using NRS scores for the study period. The ordinate shows the self-assessment of pain levels with the NRS for each day. The study days are shown on the abscissa. Treatment began on day 15 and continued at a double dose from day 29. Before IgY therapy, pain had been poorly controlled with control with carbamazepine in a daily dose of 1200 mg, which had resulted in toxic levels of the drug in the blood. On starting IgY-therapy (day 15), the carbamazepine dose was reduced at intervals until the end of the study, down to a daily dose of 450 mg (day 42). At the end of the study the patient was symptom-free. Carbamazepine was completely discontinued thereafter.

### Example 7

Patient information: 43 years old, female

Length of illness: 9 years

Diagnoses:
1. Complex regional pain syndrome of lower right extremity
2. Idiopathic back pain
3. Exceptionally acute irritable bowel syndrome, complicated by daily uncontrolled bowel movements during stomach cramps
4. Painful bladder syndrome/interstitial cystitis (PB/IC), also with bladder cramps and uncontrolled urination

History and Local Findings:

After hallux valgus surgery on the right foot, 5 further surgical procedures were carried out as a result of lingering postoperative severe neuropathic pain presenting as a complex regional pain syndrome that would not respond to drug therapy.

The pain improved with multimodal pain therapy but the foot had limited strength.

Following unsuccessful attempts to treat the inflammation and extreme pain with drugs, (long-term antibiosis owing to indication of chronic infection, anti-inflammatory long-term medication) the existing irritable bowel symptoms became completely uncontrollable. The intestinal colic was associated with watery stools, which were mainly passed in bed at night in an uncontrolled manner. The painful bladder spasms led to loss of control of the sphincter.

Evidence of a Significant Remission (Elimination) of Bowel and Bladder Symptoms with Oral Therapy of a Hyper-Immunoglobulin Against Endotoxins (LPS) from Immunized Hen Egg Yolks (Anti-LPS Hyper-IgY):

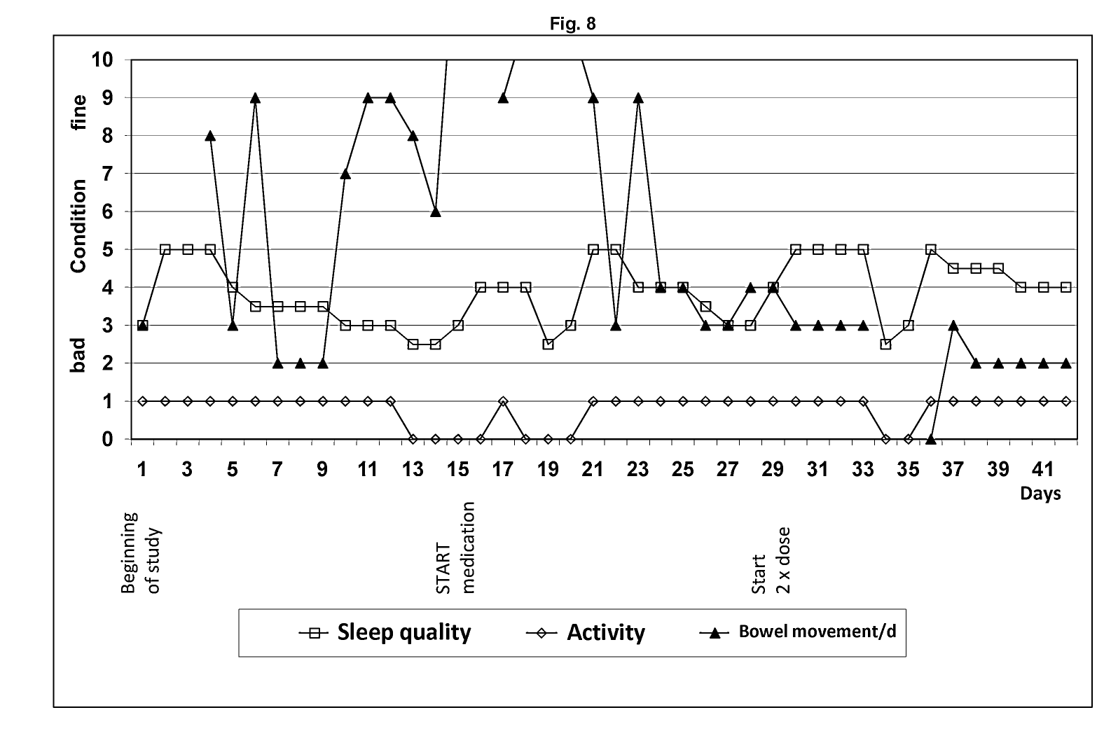

The neuropathic pain symptoms in the right foot had already improved under previous multimodal therapy (combined-care treatment, involving psychological, physical and pharmacological therapy), and only improved slightly during treatment with the sample preparation. The foot could no longer bear any load. Back pain, particularly severe in the neck and shoulder area and the lumbar-sacral region, improved by 2 points on the numerical rating scale. The bowel and bladder spasms disappeared completely towards the end of the IgY study on a daily dose of 5 g of the specific IgY preparation. The number of daily bowel movements fell from an average of 9 (2-17) to 2 (see FIG. 8), the stool no longer contained any undigested food, and was formed. Uncontrolled bowel movements and urination ceased completely.

Thereafter, the results of the treatment were maintained with a daily dose of IgY of 4 g.

In the laboratory part of the study, typical responses to treatment were found, in particular a significant reduction of endotoxin-activated monocytes in the peripheral blood (reduction through apoptosis), a significant decrease in the total number of monocytes; quantitative analysis of 22 immuno-messengers (plasma concentrations of chemokines, cytokines, growth factors) showed a significant reduction in growth factors IGF-1 and GMCSF and proinflammatory cytokines IFN-γ, TNFαR-1, IL-8 and IL-6, and an increase in anti-inflammatory cytokines IL-4, IL-5 and IL-13.

Withdrawal Study:

Several attempts were made to end treatment with IgY, but each time the bowel and bladder symptoms returned after a few days.

Summary:

Irritable Bowel Syndrome, Painful Bladder Syndrome/Interstitial Cystitis (PBS/IC), and Symptoms of an Autonomic Neuropathy

Irritable bowel syndrome of this severity is certainly a rarity, as is the combination with similar bladder symptoms. Nine years of failed attempts to treat the foot with drug therapy have resulted in considerable damage to the barrier function of intestinal mucosa and certainly contributed to the unusual extent of the disease. The over 90% endotoxin-activated blood monocytes (CD14+ and CD45+) before the IgY treatment ultimately reveal the consequences of this barrier damage, which has resulted in insufficient apoptosis of antigen-receiving monocytes in the intestine, causing an abnormally high endotoxin load in the whole body. In laboratory tests at the end of study, monocytes with endotoxin binding (CD14+) had fallen to 65% and the “activated” monocytes (CD45+) had fallen to 10%. The simultaneity of the bowel and bladder symptoms with significant dysfunction of the sphincter muscles of both organs suggests that endotoxins were the primary cause of an autonomic neuropathy. Bowel and bladder pain and dysfunction could largely be interpreted as damage to the autonomic nerve supply of both organ systems caused by endotoxins.

FIG. 8 shows the effect of the IgY preparation on 3 quality of life indicators (sleep quality, activity and bowel movements) in this patient, who had an extreme manifestation of irritable bowel syndrome with diarrhoea.

The ordinate shows the self-assessment score for “sleep quality” and “activity” using NRS daily values and the daily number of bowel movements.

The abscissa shows the time in days. The study medication was started on day 15, and continued in a double dose from day 29 to day 42.

### Example 8

Patient information: 55 years old, male

Length of illness: 19 years

Diagnoses:
1. Post-Lyme disease syndrome with signs of chronic encephalitis, status post Lyme carditis
2. Polyneuropathy
3. Irritable bowel syndrome with diarrhoea
4. Chronic fatigue syndrome (CFS)
5. Polymorphic light eruption

History and Local Findings:

Erythema chronicum migrans, following Borrelien radiculitis (Garin-Bujadoux-Bannwarth syndrome), encephalitis, and polyneuropathy. Oral and intravenous long-term antibiosis resulting in severe intestinal symptoms caused by bacterial overgrowth. Full disability because of the pain and the extreme form of CFS.

Thereafter: Onset of Polymorphic Light Eruption

Intermittent treatment of nerve pain (polyneuropathy) with polyvalent human immunoglobulins (IVIg). This therapy gave good pain control and CFS and resulted in a marked improvement in light eruption. Further improvement in light eruption after eradication of chronic Helicobacter pylori infection of the stomach (duration of improvement: 6 months).

Thereafter the IVIg no longer had a curative effect. The patient lived in a completely darkened room, with only UV-B-free artificial light. Largely bedridden, requiring home care.

Evidence of Significant Improvement of Polyneuropathy, Light Eruption, CFS and Irritable Bowel Syndrome During Treatment with the Hyper Immunoglobulin Against Endotoxin (LPS) from Egg Yolks of Immunized Hens (Anti-LPS Hyper-IgY):

Admitted to inpatient care at the Dermatological University Clinic Würzburg.

Admitted to a daylight-proof single room. Continued pain therapy (neuropathy) with gabapentin, starting treatment with the specific IgY preparation in a daily dose of 2×1.25 g. This resulted in a significant improvement in neuropathic pain and complete normalization of bowel movements. After one week, the IgY preparation dose was increased to 1.5 g 3×daily. On this treatment, there was a daily increase of daylight exposure from 300 to 9,000 lux per day until discharge for home care after 4 weeks.

Summary:

Polyneuropathy After Neuroborreliosis, Chronic Fatigue Syndrome (CFS), Irritable Bowel Syndrome, Polymorphic Light Eruption

All disease symptoms were controlled by IVIg over a 6-year period, to the extent that the patient remained unable to work but was largely self-sufficient. Even under optimal conditions, i.e. in the first 4 weeks after each intermittent treatment of 30 g of IVIg, the patient could not walk more than 500 m 3 times per day. The greatest stress was caused by increased pain, chronic fatigue syndrome, and extremely itchy inflammatory skin changes when the low light tolerance threshold was exceeded. Lesions, once they appeared, took weeks to heal. The antiepileptic drug gabapentin provided minimal relief for the itching. Antihistamines and cortisone brought no relief.

Polyneuropathy

The polyneuropathy was characterized by shooting pains when moving, which were projected onto regions of the body with reduced sensitivity, mainly in the left hemisphere. In addition, there were symmetrical pains in the 2^{nd} and 3^{rd} trigeminal limbs caused by chewing or touching of the skin (trigeminalneuropathy). During treatment with IgY, symptoms of trigeminal neuropathy disappeared completely, and other pain was reduced so much that, despite the very weak condition of the patient, activities such as getting out of bed, dressing, showering, writing, and walking short distances could be carried out without any pain.

CFS

The severe daytime fatigue, which throughout the disease was only periodically interrupted when the patient was being treated with IVIg, was alleviated by IgY to the extent that the patient was able to carry out most day-to-day activities without needing a break.

Irritable Bowel Syndrome

Irritable bowel syndrome consisted in abdominal spasms and frequent unformed bowel movements. Notably, the patient reported that he was never able to fully empty the rectum, and that for a half-hour or longer after going to the toilet small quantities of semi-liquid stool would be passed unnoticed, so he was forced to wear pads. This loss of control over bowel movements was a clinical sign of autonomic neuropathy. These symptoms disappeared in the first week of treatment with IgY.

Polymorphic Light Eruption

The polymorphic light eruption was very acute. Testing a small area of skin with a predetermined dose of UV-B produced a vigorous localized reaction with the typical dermatological results of the disease.

The response of this particularly acute clinical condition to IVIg is described in the case reports, as is the successful treatment by plasmapheresis (plasma exchange treatment).

The significant partial success of treatment with Anti-Endotoxin Hyperimmune IgY is on the one hand very surprising, and on the other provides evidence of the involvement of endotoxins in the aetiology of this individual case.

### Example 9

Patient information: 59 years old, male

Length of illness: 6 months

Diagnoses:
1. Floor of mouth squamous cell carcinoma (right side), operated, irradiated
2. Diabetes mellitus type I
3. Neutropenia, anaemia (as a result of radiotherapy)
4. Neuropathic facial pains
5. Mucositis of the irradiated oral mucosa

History and Local Findings:

Since radiotherapy of the treated area in the region of the right lower jaw/floor of mouth, the patient had experienced acute facial pain radiating from the lower jaw and the right ear, brought on by swallowing. Severe burning sensation in the oral mucosa of the irradiated area.

Resting pain largely controlled with tramadol+metamizol. Almost impossible to eat during pain episodes.

Firstly, treatment with 6.4 g of subcutaneous immunoglobulin. After just a few hours the neuropathic facial pain was alleviated, but not the local contact pains related to the oral mucositis.

Evidence of Significant Improvement of Mucositis, Unhindered Oral Intake of Food While Being Treated with Hyper Immunoglobulin Against Endotoxin (LPS) from Egg Yolks of Immunized Hens (Anti-LPS Hyper-IgY):

Immediate response of contact pain in inflamed oral mucosa on eating and drinking. Normal food intake generally restored.

Summary:

The bacterial colonization of the oral mucosa may contain endotoxin-producing bacterial populations. The sensory nerve endings of the trigeminal nerve carry binding sites for endotoxins (Toll-like receptor 4), so that endotoxins can cause extreme pain and hypersensitivity in inflammatory mucosal lesions. The binding of the endotoxin with locally administered antibodies eliminates not only the pain but also the inflammation caused by the endotoxin. Unlike local anaesthetic action, the antibodies also accelerate healing.

The above-mentioned examples all used the antibody preparation from the sample preparation 1. The treatment success for uses according to the invention is not exclusively limited precisely to this sample preparation 1. The sample preparation 2 was used in the following examples. In addition, it is probable that even better results can be achieved with alternative formulations than with the sample preparations 1 and 2 used. It is of course possible for the medical expert to adjust the composition of the preparation to particular specifications or to patients' individual needs, within the limits of the agent or preparation used. Accordingly, it should of course be clear to the medical expert that use according to the invention does not relate only to the sample preparations 1 and 2 used in the examples, but instead the surprising effects may also be anticipated in other agents or preparations to be used according to the invention.

### Example 10

Patient data: 13 years old, male

Duration of illness: 1 week

Diagnoses:

1. Acute right-side periarthritis humero-scapularis (rotator cuff tendinitis)

History and Local Findings:

For approximately 1 week, the patient has been suffering increasing pains when moving in the right shoulder joint. After 5 days, an additional night-time pain at rest occurred, and after 6 days the right arm became completely unusable. Bending the elbow joint is so painful that it is impossible for the patient to dress himself or to clench his first (triggers shoulder pain).

The boy cannot remember any triggering trauma or overloading. In the history, only allergic asthma appears but was not present at the beginning of the pain symptoms.

The right shoulder joint is extremely painful in response to pressure in the region of the entire rotator cuff. In comparison with the opposite side, a temperature increase can also be determined here. Furthermore, a slight diffuse swelling of the soft tissues around the shoulder joint can be observed as far as the region of the upper shoulder blade. The patient avoids any active movement of the arm and the hand. The passive mobility of the shoulder joint is restricted to a maximum degree because of pain being triggered in all movement axes.

These are typical symptoms of an idiopathic acute periarthritis which has previously received no treatment.

Evidence of Complete Therapy of the Periarthritis with Treatment with the Hyperimmunoglobulin Against Endotoxin (LPS) Comprising Egg Yolk of Immunised Hens (Anti-LPS-Hyper-IgY):

The therapy was carried out by administering 2×½ bags of IgY effervescent powder (daily dose; corresponds to 2×2.5 g of antibody mixture). No analgesics were prescribed or taken.

First re-examination on morning after start of therapy:

The patient had again slept through the night, was already able to dress himself independently in the morning and also fasten his shoelaces. He was able to greet the examiner by gently shaking hands, and spontaneous bending of the elbow joint was possible without triggering a substantial amount of pain in the shoulder.

Within 5 days, a continuous improvement occurred until freedom from any symptoms was achieved. A total of 7 bags of the IgY preparation were taken. The last examination of the patient took place after an additional 6 weeks. There had been no recurrence of the symptoms.

Summary:

This was acute idiopathic periarthritis of the shoulder joint without prior treatment. The IgY therapy resulted in a rapid and complete therapy which started from the first dose and was complete after 5 days.

### Example 11:

Patient data: 51 years old: male

Duration of illness: 7 years

Diagnoses:

1. Pemphigus vulgaris

History and Local Findings:

The illness has existed for 7 years. It involves a rare auto-immune illness of the skin and mucous membranes. The manifestation in the region of the mucous membrane of the mouth causes extensive losses of the mucous membrane which leaves behind extremely painful ulcers (in the sense of mucositis) which do not heal until chemotherapy of the illness takes place.

During this time, oral ingestion of food and liquid is scarcely possible.

Until now, the illness was able to be interrupted by chemotherapy in ever-increasing phases. However, all attempts to reduce the chemotherapy resulted in recurrences which generally began in the region of the oral mucosa.

Since treating the oral mucosa with IgY, it was possible to maintain oral nutrition in the last two episodes of the illness because the pain already decreased substantially a few hours after the start of the ingestion.

In the last few days, the patient has again observed small areas of painful mucous membrane damage in the mouth, an unmistakeable sign of a repeated occurrence of the illness.

Individual Therapeutic Attempt with IgY:

At first, local symptom therapy with IgY effervescent powder took place at a dose of 2×1.25 g per day ((IgY preparation comprising sample preparation 2) until the ingestion of food was possible again without impediment and without pain (maximum one week).

Subsequently, treatment began with the enteric-coated administration form of the IgY preparation with the intention of eliminating LPS as a possible trigger of the system illness already in the region of the small intestine.

Dose: 3×3 enteric-coated tablets daily for the period of one month (corresponds daily to almost 3.4 g IgY preparation comprising sample preparation 2).

During this time, the oral treatment continued with IgY effervescent powder in a minimum dose in order to maintain the intact mucous membrane in the mouth and throat.

The material requirement is, in the first month: daily 3×3 enteric-coated tablets (almost 3.4 g daily dose of IgY preparation comprising sample preparation 2). The patient was prescribed 270 enteric-coated tablets and 37 daily dose units of effervescent powder.

The decision regarding continuation of the treatment at the same dose or a different dose is made at the end of each monthly period. Control of the therapy effect is carried out by diary entries concerning the symptoms of the illness, progression and dosage of the immunosuppressive chemotherapy.

This involves the first attempt to treat a patient suffering from Pemphigus vulgaris with IgY. The treatment of this patient's mucositis (oral mucosa) has been successful with each application in the past.

Further Progression of the Illness with Treatment with IgY (as at 10.03.2012):

After the painful mucous membrane lesions of the mouth/throat area were reduced by administering IgY effervescent powder, a substantial improvement in the general situation occurred just 4 days after the start of the ingestion of the enteric-coated tablets (3×3 tablets daily):
Complete elimination of the chronic exhaustion symptoms
Restored physical endurance
Elimination of non-specific joint pains in the region of the shoulder girdle

During the next analysis of the specific antibody titre (auto-antibody) in the University Clinic for Dermatology, the lowest titre since the beginning of the illness was measured (1:300). Before the treatment, this titre was >1:10,000.

After taking blood to determine the immunological activity parameters under a clinical optimum state, the IgY therapy was terminated after 6 months.

After almost 4 months' break in therapy, at the end of February 2012 symptoms of Pemphigus vulgaris occurred again for the first time (oral mucosa lesions and blisters in the region of the skin of the upper body region). Non-specific joint pain and slight exhaustion symptoms preceded the relapse of the auto-immune illness.

Blood was again taken for analysis of the auto-antibodies and the immunological activity parameters. The titre of specific auto-antibodies had increased only slightly (1:400).

Treatment with IgY was begun again. One bag of effervescent powder per day and 3×3 enteric-coated tablets were administered (corresponds to a daily dose of almost 8.4 g of the IgY preparation comprising sample preparation 2).

The non-specific pain symptoms disappeared within a few days and the (slightly pronounced) erosions of the oral mucosa quickly healed. No new blisters appeared in the region of the skin and the old ones healed within 14 days.

In this Progression, Without Doubt a Healing Effect of the IgY Preparation on the Overall Symptoms of the Auto-Immune Illness Can Now be Recognised.

The recurrence of the illness after a 4-month break in therapy was able to be inhibited first without the use of dexametasone and mycophenolate-mofetil.

### Example 12

Patient data: 41 years old, female

Duration of illness 9 months (after bone marrow transplant (BMT) owing to leukaemia)

Diagnosis:

1. Chronic graft-versus-host disease (GvHD)

History and Local Findings:

Nine months after the BMT, a chronic GvH of the mouth and genital mucous membranes developed and a GvH kerato-conjunctivitis. A short time later: acute lung involvement of the GvH with general insufficiency of the lungs necessitating respiration. After survival of the lung GvH, long-term therapy occurred with prednisolon at a dose between 20 and 30 mg daily in addition to the chemotherapy for the leukaemia. The cortisone administered resulted in pronounced Cushing's syndrome.

The chronic GvH of the mouth and eyes and the vaginal mucous membranes does not allow any reduction in the dose of cortisone below 20 mg. The lungs are not affected currently but are still subjected to significant function limitations.

From March/April 2011, the oral IgY therapy began with daily administration of 2 teaspoons of powder (corresponds to approximately 2.5 g of IgY preparation comprising sample preparation 2) in a vanilla yoghurt. An improvement in the mouth and eye involvement but not in the genital GvH symptoms was observed. The administered quantity of prednisolon was able to be reduced to a 15 mg daily dose.

Subsequently, a “wash-out” phase took place for the period of one month.

Individual treatment plan of the therapeutic attempt with enteric-coated IqY tablets and (optionally) IqY effervescent powder:

The treatment plan provides for the daily administration of 3×3 enteric-coated tablets (almost 3.4 g daily dose) in the first month initially for a period of 4 weeks. Patients are prescribed 270 enteric-coated tablets of IgY for the first month.

All the symptoms of the GvH are recorded. After discussions with the acting oncologist and in accordance with the clinical findings, the intention is to reduce the administered dose of corticoid and accordingly the immunosuppression. In the case of clinical remission of the GvH symptoms, the therapy is continued at the same dose until complete withdrawal of the cortisone therapy. In the case of incomplete remission of the symptoms or necessity to maintain the cortisone medication, the additional ingestion of effervescent powder is provided for in the next step for the period of an additional 4 weeks. Patients are prescribed 270 enteric-coated tablets of IgY for the second month.

The treatment plan further provides for the administration of an identical dose of enteric-coated tablets (3×3 tablets; almost 3.4 g daily of sample preparation 2) in the second month for the period of another 4 weeks and the additional administration of 2×½ bag of effervescent powder (5 g IgY preparation comprising sample preparation 2) (based on the daily dose in each case). The intention is to examine whether the oral effect produces an additional advantage for the oral manifestation, optionally also the conjunctival manifestation. Patients are prescribed 270 enteric-coated tablets and a further 30 bags of effervescent IgY powder for the second (and where applicable third) month.

If a non-optimum overall effect occurs, the dose of the enteric-coated IgY tablets is intended to be increased for the period of an additional 4 weeks to 3×4 tablets (daily dose of 4.5 g IgY preparation comprising sample preparation 2) and additional effervescent powder only where an advantage is assumed. Patients are prescribed 360 enteric-coated tablets and a further 30 bags of effervescent IgY powder for the fourth month.

If at any period of at least one month a complete remission of the GvH results without cortisone medication, the dose is intended to be reduced in weekly steps by 3×1 tablet (daily dose) until the maintenance dose is reached. The maintenance dose must then be established.

The corner times for taking blood samples (if necessary also stool samples) are:
1. At the end of the “wash out” phase and before the start of the first four week period, during which the enteric-coated IgY tablets are administered.
2. After the first 4-week period
3. After the cortisone has been stopped
If a remission occurs
If clinical symptoms return with a reduction in dose

If the effect is positive, the patient may continue to receive the preparation at a dose according to need. In the case of complete remission of the symptoms over a period of 2 months, an attempt will be made to withdraw the therapy.

The symptoms of GvH were recorded by the patient from the beginning of March 2011 in a journal according to the Visual Analogue Scale (pain), and the visible symptoms were documented by a medical expert.

This was the first attempt to treat chronic GvH with IgY.

Results of this Individual Therapy Attempt:

Owing to an unforeseen increase in the tumour markers in April 2011 and the associated need to stop cortisone treatment quickly, the first blood sample was taken 4 weeks after the end of the test phase with IgY effervescent powder. The cortisone medication had already been discontinued by this point and treatment with IgY was then started, counter to the original treatment strategy, with 3×4 tablets of the enteric-coated formulation combined with a bag of effervescent powder.

The acute increase in the tumour markers was a typical consequence of the high dose of cortisone medication which was necessary to suppress the GvHD. Cortisone inhibits not only the graft versus host reaction (GvHR) but also the anti-tumour activity of the donated bone marrow to the same extent (inhibition of the graft versus tumour activity).

This treatment led to a constant improvement in all illness symptoms of the GvHD (despite discontinuation of the cortisone). The tumour markers were soon unable to be detected in the blood. The chemotherapy was therefore gradually decreased to a minimum dose. In autumn 2011, the patient resumed work after 1½ years, and in January 2012 blood was taken for analysis of the immunological activity parameters for a second time at a point at which the patient had almost completely recovered all functions.

IgY therapy is continued in a combination of the effervescent powder with the enteric-coated tablets with a slow reduction of the dose. Should the positive state continue to remain stable, a complete termination of oncological pharmacotherapy is planned.

### Example 13

Patient data: 55 years old, male

Duration of the illness: 18 months

Diagnoses:

1. Chronic Epicondylitis humeri radialis on both sides (more pronounced on right side)

History and Local Findings:

The patient is a sports teacher, swimming team trainer and sports therapist in a physio-therapy practice. The patient has been suffering from epicondylitis for 18 months, at first purely on the right-hand side, as the illness progressed on both sides with the right-hand side being more pronounced, always limited to the radial epicondylus.

Previous treatment was carried out in an orthopaedic practice. Oral drugs inhibiting inflammation did not produce any improvement. Local injections with local anaesthetics and cortisone resulted in improvements which had been lasting a maximum of one day for some time. Physiotherapy, tapes and other auxiliary media were unable to have an impact on the advancement of the symptoms.

Individual Therapy Attempt with IgY:

The individual therapy attempt with IgY began when night-time pain at rest did not allow permit coherent night-time sleep and matutinal weakness when clenching the first (both sides) for a period of approximately 1 hour meant the patient was no longer able to work.

The patient did not have any other illness symptoms, and it was the patient's first pain syndrome.

The IgY therapy was started with the effervescent powder preparation at a daily dose of 1 bag (5 g IgY preparation comprising sample preparation 2).

During the first week of treatment there was no improvement of the symptoms.

Only in the second week was there a significant reduction in pain to approximately half of the starting level and pain at rest only rarely caused wakefulness at night.

This improvement continued for approximately 3 weeks until, after an infection of the upper airway (bronchitis, maxillary sinus inflammation), the pain increased again. Subsequently, additional IgY therapy started with enteric-coated tablets (at a dose of 2×4 tablets; corresponds to 3 g IgY preparation comprising sample preparation 2).

Using this combination, for the first time there was an almost complete elimination of the symptoms. The patient reported waking in the morning without any stiffness of the fingers, full stability of the radial lower arm muscles and no pain at night.

There were no longer residual symptoms everyday, wherein the residual symptoms were primarily a sensitivity to impacts of the elbow. High-performance sport (cross-country skiing) is possible without limitation with continuation of the therapy.

Two blood samples were taken to analyse the immunological activity parameters, once before the start of the treatment and once in the state of substantial freedom from symptoms.

1. Antibody products with n-specific antibodies
characterized in that
a) the n-specific antibodies in each case have an antibody content of at least 6/n % by weight of the total antibody component of the antibody product, and
b) 2, 3 or more of the n-specific antibodies target lipopolysaccharide-expressing microorganisms, and
c) the total amount of n-specific antibodies is ≧7% by weight of the total antibody content of the antibody product.

2. Antibody products in accordance with claim 1, characterized in that a proportion of ≧50%, preferably ≧60%, most preferably ≧70% by weight of the total content of n-specific antibodies is targeted against lipopolysaccharide-expressing microorganisms.

3. Antibody products in accordance with any one of the preceding claims, characterized in that the antibody product is a drug.

4. Antibody product in accordance with any one of the preceding claims, characterized in that n≦10.

5. Antibody product in accordance with any one of the preceding claims, characterized in that each of the n-specific antibodies independently target microorganisms selected from the group consisting of:
a) Gram-negative bacteria, preferably selected from the group consisting of Streptobacillus moniliformis, meningococcus, Chlamydophila, chlamydia, spirochetes, cyanobacteria, species of the Proteobacteria strain, especially Enterobacteriaceae (Escherichia coli, Salmonella, Shigella, Klebsiella, Proteus, Enterobacter), Pseudomonas bacteria, Legionella bacteria, Neisseria bacteria, rickettsia bacteria, Pasteurella multocida bacteria and species of the Bacteroidetes strain, and
b) Bacteria that cause food poisoning and
c) Inflammatory agents and/or
d) optionally other microorganisms.

6. Antibody products in accordance with any one of the preceding claims, characterized in that the n-specific antibodies contain at least one specific antibody against:
a) Clostridium perfringens Type C,
b) F 18 Escherichia coli and
c) Salmonella typhimurium.

7. Antibody products in accordance with any one of the preceding claims, characterized in that the antibody product does not contain lactose.

8. Antibody products in accordance with any one of the preceding claims, characterized in that the antibody is or forms part of a prepared formulation for administration, where the prepared formulation is selected from the group consisting of pharmaceutical preparations, cosmetic preparations, foodstuffs, food supplements, functional food and medical products, as well as animal feed, feed supplements and dietary supplements.

9. Antibody products in accordance with any one of the preceding claims, characterized in that the antibody product is prepared for oral treatment.

10. Method for producing an antibody product in accordance with any one of claims 1 to 9, comprising the following steps:
a) Immunizing n groups of animals with only one micro-organism species each and/or a part of the only one microorganism species, where each n group is given a different microorganism species and/or part of the different micro-organism species, and where at least 2 of the micro-organism species and/or parts of the microorganism species are lipopolysaccharide-expressing micro-organism species or are derived therefrom.
b) Obtaining an antibody-containing fraction from each of the n groups,
c) Mixing the antibody fractions,
d) If necessary, concentrating the antibody content in the antibody fractions and/or in the mixture of antibody fractions.

11. Antibody product which can be or is produced using a method in accordance with claim 10.

12. Antibody product in accordance with claim 11, wherein each specific antibody is contained in the antibody product at a proportion of 6/n % by weight in relation to the total antibody proportion of the antibody product.
